(19) United States
(12) Patent Application Publication
(10) Pub. No.: US 2014/0219921 A1
(43) Pub. Date: Aug. 7, 2014

(54) IMMUNOMODULATING COMPOSITIONS AND METHODS

(71) Applicant: Rutgers, The State University of New Jersey, New Brunswick, NJ (US)

(72) Inventor: Pranela Rameshwar, Maplewood, NJ (US)

(73) Assignee: Rutgers, The State University of New Jersey, New Brunswick, NJ (US)

(21) Appl. No.: 14/139,394

(22) Filed: Dec. 23, 2013

Related U.S. Application Data
(60) Provisional application No. 61/740,613, filed on Dec. 21, 2012.

(51) Int. Cl. A61K 35/28; A61K 49/00

(52) U.S. Cl. CPC A61K 35/28; A61K 49/0004; A61K 2035/122
USPC 424/9.2; 424/278.1

(57) ABSTRACT

The disclosure provides compositions in the form of cells exhibiting the phenotype of a mesenchymal stem cell and uses of such cells in the preparation of medicament and the treatment of an allergic reaction in an asthmatic subject. Also provided are methods of inducing tolerance to an allergen in an asthmatic subject and methods of assessing a non-asthmatic subject for amenability to allergy treatment by administering mesenchymal stem cells.

### CROSS REFERENCE TO RELATED APPLICATION

This application claims the priority benefit of provisional U.S. Patent Application No. 61/740,613 filed Dec. 21, 2012, which is incorporated by reference herein in its entirety.

### FIELD

The disclosure relates to compositions and methods of using mesenchyme stem cells in the treatment of inflammatory disorders and conditions.

### BACKGROUND

Allergic rhinitis (AR) is a common chronic inflammatory disease of the nasal passages, affecting approximately 20% of adults in the United States. AR results from antigen-induced, IgE-mediated mast cell degranulation, which is characterized by an influx of eosinophils and Th2 cells. Typical symptoms include chronic nasal congestion, rhinorrhea, sneezing, postnasal drip and itchy and watery eyes, which occur as a result of immune hyper-reactivity to common environmental allergens. Significant morbidity results from AR, particularly when it coexists with asthma. Uncontrolled AR can result in worsening of other conditions, such as asthma and sinusitis, and increase health care utilization and cost. Despite treatment, many patients suffer from decreased quality of life, impaired productivity, and missed workdays.

Mesenchymal stem cells (MSCs) differentiate along various lineages to generate specialized cells of all germ layers, such as cartilage, muscle, neurons and cardiomyocytes. These ubiquitous multipotent cells are abundant in adult bone marrow and adipose tissue. MSCs can be found in fetal organs and amniotic fluid. Over the last decade, MSCs have gained significant interest because of their pliability to develop into almost any cell type, making them attractive for regenerative medicine, including the fields of orthopedic surgery, rheumatology, gastroenterology, and transplant surgery. MSCs also have potential as cellular immune-suppressive therapy for inflammatory disorders, such as systemic lupus erythematosis, rheumatoid arthritis, inflammatory bowel disease, and graft-versus-host disease.

Mesenchymal Stem Cells (MSCs) can suppress and enhance immune functions. Due to the presence of several receptors on MSCs, including those for cytokines, the functions of MSCs can be influenced by the tissue microenvironment through cross-communication with specific ligands. Allergic asthma is a heterogeneous, antigen-driven disorder in subjects with aberrant T-cell regulation. Because allergic asthma is a heterogeneous disorder; it would be difficult to predict how MSCs would respond, in vivo.

In view of the foregoing, it is apparent that needs continue to exist in the art for compositions and methods of treating inflammatory conditions, such as allergic reactions, that are efficacious, non-toxic, not plagued by deleterious side effects and cost-effective.

### SUMMARY

The disclosure provides compositions and methods that satisfy at least one need in the art by providing a therapeutic in the form of mesenchymal stem cells useful in inhibiting or reducing, and thereby treating, inflammatory conditions, e.g., in asthmatic subjects. Also provided is a method of inducing tolerance to an allergen by preconditioning the peripheral blood mononuclear cells of a subject with exposure to mesenchymal stem cells and/or by administering at least one low or sub-acute, i.e., sub-stimulatory, dose of an allergen in combination with mesenchymal stem cells, to a subject at risk of an allergic reaction. Further provided is a method of assessing whether a subject is amenable to anti-inflammatory treatment using mesenchymal stem cells. In all of these methods, the subject may be a human or an animal such as a non-human mammal, such as a domesticated animal or a pet.

One aspect of the disclosure provides a method of treating an inflammatory condition in an asthmatic subject comprising administering an effective dose of a cell exhibiting the phenotype of a mesenchymal stem cell. In some embodiments of the method, the inflammatory condition is systemic lupus erythematosis, rheumatoid arthritis, inflammatory bowel disease, Crohn's disease, autoimmune enteropathy, colitis, neonatal colitis, neonatal necrotizing enterocolitis, pouchitis, or an allergic reaction. An exemplary method is one wherein the inflammatory condition is an allergic reaction in the subject. In some embodiments of this aspect of the disclosure, the cell is a mesenchymal stem cell, which may be an autologous cell. The mesenchymal stem cell is, or may be, obtained from bone marrow, adipose tissue, placenta, fetal organ, or amniotic fluid.

In some embodiments of the above-described method, the allergic reaction is a reaction to dust mite, e.g., Dermatophagoides farinae or Dermatophagoides pteronyssinus. In some embodiments, the allergic reaction is a reaction to a food allergen, a drug allergen, or an environmental allergen. Exemplary methods include methods wherein the food allergen is fruit, garlic, oats, meat, a milk product, peanut, fish, shellfish, soy, a tree nut, wheat, gluten, egg, a sulfite, carbohydrate, or chocolate, or the drug allergen is tetracycline, dilantin, tegretol, penicillin, a cephalosporin, a sulfonamide, a non-steroidal anti-inflammatory drug (NSAID), an intravenous contrast dye, or a local anesthetic, or the environmental allergen is pollen, pet dander, an insect bite, an insect sting, mold, perfume, a cosmetic, semen, latex, metal, formaldehyde, tobacco, or a photographic developer.

Another aspect of the disclosure is a method of inducing tolerance to an allergen in an asthmatic subject comprising: (a) administering an effective dose of mesenchymal stem cells; and (b) delivering at least one sub-acute, i.e., sub-stimulatory, dose of an allergen to the subject, thereby inducing tolerance to the allergen in the subject. In some embodiments of this aspect of the disclosure, the sub-acute dose of an allergen produces 0.125-5.0 μg/ml trough concentration in serum. In some embodiments, the mesenchymal stem cell is an autologous cell, which is, or may be, obtained from bone marrow, adipose tissue, placenta, fetal organ, or amniotic fluid. In some embodiments, the allergic reaction is a reaction to dust mite, e.g., wherein the dust mite is Dermatophagoides farinae or Dermatophagoides pteronyssinus.

Yet another aspect of the disclosure is a method of assessing a subject for amenability to allergy treatment with a cell exhibiting the phenotype of a mesenchymal stem cell comprising: (a) contacting a non-asthmatic subject with at least one dose of an allergen; and (b) determining the response of the subject to the allergen, wherein an acute allergic reaction is indicative of an individual amenable to allergy treatment with the cell.

Other features and advantages of the present disclosure will become apparent from the following detailed description, including the drawing. It should be understood, however, that the detailed description and the specific examples, while indicating preferred embodiments, are provided for illustration only, because various changes and modifications within the spirit and scope of the disclosure will become apparent to those skilled in the art from the detailed description.

### BRIEF DESCRIPTION OF THE DRAWING

FIG. 1 presents a flow diagram indicating inclusion and exclusion criteria for enrolling subjects in the study.

FIG. 2 presents data from studies relating to the specificity of the DM antigen. A) Dose-response studies were performed with PBMCs from DM(+) subjects in a proliferation assay. The data [disintegrations per minute (dpm)] are presented for three subjects, each tested in independent studies, mean±SD, n=9. *p<0.01 vs. other concentrations of DM. B) Time-course studies were performed with the optimum (5 μg/mL) DM, as described in ‘A’. C) Proliferation assays were performed in the presence of DM and/or MSCs with optimum DM. Four subjects were studied in each category and each studied in three independent experiments. The data are presented as mean±SD, n=12.

FIG. 3 presents data on the effect of MSCs on the proliferation of DM-/tetanus toxoid-challenged PBMCs. PBMCs were assessed for proliferation in the presence or absence of DM, n=7 (A) or tetanus toxoid, n=4 (B) and/or MSCs. Each subject was assessed in three independent experiments and the data are presented as Stimulation Index (S.I.)±SD. *p<0.05 vs. DM+MSCs.

FIG. 4 presents flow cytometry data on levels of T_{regs} and DCs in DM-challenged PBMCs and MSCs. Flow cytometry was performed for T_{regs} (A) and DCs (B) in multi-color flow cytometry with PBMCs from subjects with allergic asthma and healthy subjects challenged with DM in the presence or absence of MSCs for five days. In the case of DC, cells negative for CD64 were analyzed for CD86 and HLA-DR expression within the CD 14+ and CD 14− subsets. The results are shown for allergic asthma, representing three experiments of all sub-groups.

FIG. 5 presents data showing the loss of response to DM by preconditioning PBMCs from allergic asthma subjects with MSCs. PBMCs from DM sensitivity (n=4) and/or asthma (n=4) were pre-incubated with MSCs. At day five, the MSC-free PBMCs were studied in DM-challenged mitogen assays. Each subject was studied in three independent experiments. The results are presented as S.I.±SD (n=12). *p<0.05 vs. preconditioned PBMCs without DM.

FIG. 6 presents data showing refractoriness of PBMCs from allergic asthma subjects following repeated challenge with low dose DM and MSCs. PBMCs were challenged with low dose DM in the presence of MSCs, as outlined in the Method section. Parallel studies were performed in which PBMCs were challenged once with optimal DM in the presence or absence of MSCs. Cells with repeated DM challenges were washed and then re-exposed to optimal DM in proliferation assay. The results represent studies with four subjects (S1-S4), each performed in three independent experiments.

FIG. 7 shows the proliferative response of PBMCs to rye grass and/or MSCs. Peripheral blood (PB) from subjects with allergy to rye grass was taken out of season and the PBMCs were challenged with 5 μL/mL in the presence or absence of MSCs. After 4 days, the cultures were assessed for proliferation by tritiated thymidine uptake. Each of three subjects was studied in three independent experiments and the data are presented as the mean±SD, n=9.

FIG. 8 shows the proliferative response of PBMCs to ragweed and/or MSCs. The proliferation of PBMCs from subjects with allergy to ragweed was studied as for FIG. 1 with optimum ragweed. Four subjects were studied, each in three different independent experiments and the data are presented as the mean±SD, n=12.

FIG. 9 shows the proliferative response of PBMCs to ragweed and/or MSCs. The studies in FIG. 2 were repeated with PBMCs from a subject with high baseline proliferation to ragweed. The studies were performed in three independent experimental time points and the data presented as the mean±SD, n=3.

FIG. 10 presents the results of APC function by MSCs, in the presence of ragweed and PBMCs from AR subjects. MSCs were studied for APC function with PBMCs. Each experimental point was performed in three independent studies and the data presented as mean±SD, n=12.

FIG. 11 describes the effects of CIITA knockout MSCs in the APC response to ragweed. A) MSCs were knockdown for CIITA with different clones of shRNA. Western blots were performed for CIITA and MHC-II (HLA-DR) and normalization with anti-β-actin. B) APC assay was performed as for FIG. 4 with MSCs, untransfected, transfected with vector alone or knockdown for CIITA. The results are presented at the mean±SD, n=4.

FIG. 12 summarizes the difference in responses by AA and AR in the presence of MSCs. Cartoon was established with the relative proliferation of PBMCs to antigen and presented as a function of the biological response of MSCs.

FIG. 13. Flow diagram on inclusion/exclusion criteria. Shown are the methods used in enrolling subjects for the study.

FIG. 14. Specificity of DM antigen. A) Dose-response studies were performed with PBMCs from DM(+) subjects in proliferation assay. The data (dpm) are presented for three subjects, each tested in independent studies, mean±SD, n=9. *p<0.01 vs. other concentrations of DM. B) Time-course studies were performed with the optimum (5 μg/mL) DM, as described in ‘A’. C) Proliferation assays were performed in the presence of DM and/or MSCs with optimum DM. Four subjects were studied in each category and each studied in three independent experiments. The data are presented as mean±SD, n=12.

FIG. 15. Effects of MSCs on the proliferation of DM-/tetanus toxoid-challenged PBMCs. PBMCs were assessed for proliferation in the presence or absence of DM, n=7 (A) or tetanus toxoid, n=4 (B) and/or MSCs. Each subject was assessed in three independent experiments and the data are presented as S.I.±SD. *p<0.05 vs. DM+MSCs.

FIG. 16. T_{reg} and DC in DM-challenged PBMCs and MSCs. Flow cytometry was performed for T_{regs} (A) and DCs (B) in multi-color flow cytometry with PBMCs from subjects with allergic asthma and healthy subjects challenged with DM in the presence or absence of MSCs for five days. In the case of DC, cells negative for CD64 were analyzed for CD86 and HLA-DR expression within the CD14+ and CD14− subsets. The results are shown for allergic asthma, representing three experiments of all sub-groups.

FIG. 17. Non-responsiveness to DM by preconditioned PBMCs from allergic asthma subjects. PBMCs from DM sensitivity (n=4) and/or asthma (n=4) were pre-incubated with MSCs. At day five, the MSC-free PBMCs were studied in DM-challenged mitogen assays. Each subject was studied in three independent experiments. The results are presented as S.I.±SD (n=12). *p<0.05 vs. preconditioned PBMCs without DM.

FIG. 18. Refractoriness of PBMCs from allergic asthma subjects following repeated challenge with low dose DM and MSCs. PBMCs were challenged with low dose DM in the presence of MSCs, as outlined in the Method section. Parallel studies were performed in which PBMCs were challenged once with optimal DM in the presence or absence of MSCs. Cells with repeated DM challenges were washed and then re-exposed to optimal DM in proliferation assay. The results represent studies with four subjects, each performed in three independent experiments.

FIG. 19: Proliferative response of PBMCs to rye grass and/or MSCs. PBMCs were isolated from the peripheral blood of subjects with AR, and sensitivity to rye grass. The PBMCs were challenged with 5 μL/mL rye grass, in the presence or absence of MSCs. After 48 h, the cultures were assessed based on the incorporation of tritiated thymidine. The proliferation for each experimental point is presented as stimulation index (S.I.). The S.I. was calculated as the disintegration per min (dpm) in the experimental point/dpm of unstimulated PBMCs alone. The results (mean±SD) are presented for six donors (Table 3, subjects S1-S6). Each AR subject was studied with MSCs from a different donor.

FIG. 20: Proliferative response of PBMCs from AR to ragweed in the presence or absence of MSCs. PBMCs were isolated the peripheral blood of subjects with AR and sensitivity to ragweed. The PBMCs were challenged with 5 μL/mL ragweed, in the presence or absence of MSCs. After 48 h, the cultures were assessed for proliferation, based on the incorporation of tritiated thymidine. The proliferation is presented as stimulation index (S.I.), which was calculated as the disintegration per min (dpm) of the experimental point/dpm of unstimulated PBMCs alone. The results (mean±SD) are presented for six donors (Table 3, subjects S5-S8, S12, S13). Each AR subject was studied with MSCs from a different donor.

FIG. 21: Proliferation of PBMCs from allergic (ragweed) asthma, in the presence or absence of MSCs. PBMCs from subjects with allergy to ragweed and asthma (allergic asthma) were stimulated with ragweed (5 μL/mL), in the presence or absence of MSCs. After 48 h, the cultures were assessed for proliferation by pulsing with tritiated thymidine. The incorporation of tritiated thymidine was detected with a scintillation counter. The proliferation for each experimental point is presented as stimulation index (S.I.), which was calculated as the disintegration per min (dpm) of the experimental point/dpm of PBMCs alone. The results (mean±SD) are presented for four donors (Table 3, subjects S18-S21). Each donor was studied in quadruplicates, with MSCs from a different donor.

FIG. 22: Antigen-Presenting Cell (APC) function of ragweed-challenged MSCs. MSCs were studied for APC function with PBMCs from AR subjects with sensitivity to rye grass (A) or ragweed (B). The PBMCs were challenged with the respective antigen for 5 days. The CD4+ cells were selected from the activated PBMCs and then added to 16-h antigen pulsed MSCs. After 16 h, the proliferation of the CD4+ T-cells was studied by tritiated thymidine incorporation. Each antigen was studied with six donors (Table 3, Rye grass, subjects S1-S4, S6, S7; Ragweed, subjects S9-14, S16). The data are presented as the S.I. (mean±SD), which was calculated by dividing the dpm of each experimental point/dpm of unstimulated PBMCs.

FIG. 23: Cytokine production and expressions of MHC-11 and CD86 in MSCs. (A) APC assays were established with activated PBMCs and pulsed or unpulsed MSCs. The cells were obtained from three subjects with AR and ragweed sensitivity (Table 3, subjects S8, S13, S22). After 48 hours, the media were analyzed in duplicate with cytokine protein arrays. The densities of the background cytokines (PBMCs alone) were subtracted from the experimental points and then presented as fold change of pulsed MSCs/unpulsed MSCs, ±SD, n=6. (B) Pulsed and unpulsed MSCs from the APC assays were studied for MHC-II and CD86 by flow cytometry using donors S17, S19 and S22 (Table 3). The analyses were done on CD105+/CD3−/CD25−. *p<0.05 vs. other cytokines.

FIG. 24: Effects of the Class II major histocompatibility complex TransActivator (CIITA) knockout Mesenchymal Stem Cells (MSCs) in the APC response to ragweed. A) MSCs were transfected with different CIITA shRNA constructs. MSCs were transfected with different shRNA constructs in an effort to knockdown expression of CIITA. Western blots were performed for CIITA and MHC-II (HLA-DR) and normalized with anti-β-actin. The membrane was stripped and reprobed after blotting with each antibody. B) APC assay was performed as for FIG. 4 with MSCs, untransfected, transfected with vector alone, or transfected with the vector encoding the shRNA construct exhibiting the optimal knockdown of CIITA. The MSCs were co-cultured with PBMCs from AR subjects with sensitivity to rye grass (Table 3, subjects S5, S7, S8) or ragweed (Table 3, subjects S14, S15, S17). CD4+ cells were selected from 5-day ragweed (5 μl/mL) challenged PBMCs and then incubated with 16-h pulsed MSCs. After 16 h, the proliferation of the CD4+ T-cells was assessed, based on tritiated thymidine incorporation. The results for ragweed and rye grass were similar and are plotted together as S.I., mean±SD, n=6. The S.I. was calculated by the dpm of each experimental point/dpm of PBMCs alone.

FIG. 25: A cartoon depicts the relative proliferation of PBMCs to antigen as a function of AR and AA. The cartoon combines the findings of this study and our previous report, which showed a suppressive effect of MSCs for allergic asthma²⁰.

FIG. 26: Proliferation studies with non-irradiated MSCs. PBMCs were isolated from the blood of subjects with allergy to rye grass or ragweed and then challenged with the optimal concentrations of each antigen (5 μL/mL), in the presence or absence of γ-irradiated or non-irradiated MSCs. After 48 hours, the cultures were assessed for proliferation by tritiated thymidine uptake. The results (mean±SD) are presented for six independent experiments, each performed with MSCs from a different donor.

FIG. 27: Dose-Response studies for rye grass-challenged PBMCs, in the presence or absence of MSCs. PBMCs were isolated from the peripheral blood (PB) of subjects with allergy to rye grass and then challenged with different amounts of rye grass, in the presence or absence of MSCs. The results are presented as the S.I. (mean±SD) for four independent experiments, each performed with MSCs from a different donor.

FIG. 28: Dose-Response studies for ragweed-challenged PBMCs, in the presence or absence of MSCs. PBMCs were isolated from the peripheral blood (PB) of subjects with allergy to ragweed and then challenged with different amounts of ragweed, in the presence or absence of MSCs. The results are presented as the S.I. (mean±SD) for four independent experiments, each performed with MSCs from a different donor.

FIG. 29: Proliferative response of PBMCs to different grasses and/or MSCs. PBMCs were isolated the PB of subjects with allergy to Timothy, Bermuda and Meadow Fescue. Each allergen was added at 5 μL/mL from stocks of 10,000 BAU/mL. The stimulations were done in the presence or absence of MSCs. After 48 hours, the cultures were assessed for proliferation by pulsing with tritiated thymidine. The incorporation of tritiated thymidine was assessed in a scintillation counter. The proliferation is presented as disintegration per minute (dpm), mean±SD, n=4.

### DETAILED DESCRIPTION

The disclosure provides MSCs having immune suppressive properties useful in treating patients to suppress the immune response. In addition, the disclosure provides MSCs and a sub-stimulatory (sub-acute) dose of allergen to induce tolerance to the allergen. A further aspect of the disclosure is a method of administering MSCs to enhance an immune reaction, as disclosed herein.

In the methods according to the disclosure, the MSC compositions are administered by any means known in the art to be suitable for delivery of cells to a subject, including but not limited to, injection or infusion by any known route of administration, such as subcutaneous, intravenous, parenteral, intramuscular, intracisternal, intrathecal, intradermal, intramammary, intraperitoneal, retrobulbar, or intrapulmonary, or by surgical implantation at a particular site. An MSC composition according to the disclosure is administered in at least one dose to prevent, treat or ameliorate a symptom of an inflammatory condition or disorder. It is within the skill in the art, and contemplated by the disclosure, that those of skill in the art, such as medical clinicians, will determine the MSC dosage on a case-by-case basis, guided by relevant considerations well known in the art, such as the general health of a subject, medical history, weight, condition or disorder to be prevented or treated, or a symptom of which is to be ameliorated. In general MSC dosages in the range of 10⁷-10¹⁰, including but not limited to 10⁸ to 10⁹ cells. In methods of inducing tolerance to an allergen, sub-acute or sub-stimulatory doses of allergen are contemplated, i.e., contemplated are doses of an allergen that do not induce a detectable immune response in terms of the appearance of characteristic symptoms of allergy (e.g., runny nose, watery eyes, itchiness). Suitable doses are determined on a case-by-case basis using criteria well known to those of skill in the art, such as the general health of a subject, medical history, weight, and the sensitivity of the subject to the allergen. An allergen may be delivered by any of the routes of administration noted above, or by oral, topical, transdermal, by inhalation spray, vaginally, rectally, or by intracranial injection. The term parenteral as used herein includes subcutaneous injections, intravenous, intramuscular, intracisternal injection, or infusion techniques. Generally, compositions are essentially free of pyrogens, as well as other impurities that could be harmful to the recipient.

Pharmaceutical compositions of the disclosure containing MSC, or containing MSC and a sub-stimulatory dose of allergen, for use in a method of the disclosure may contain pharmaceutically acceptable carriers or additives depending on the route of administration. Examples of such carriers or additives include water, a pharmaceutical acceptable organic solvent, collagen, polyvinyl alcohol, polyvinylpyrrolidone, a carboxyvinyl polymer, carboxymethylcellulose sodium, polyacrylic sodium, sodium alginate, water-soluble dextran, carboxymethyl starch sodium, pectin, methyl cellulose, ethyl cellulose, xanthan gum, gum Arabic, casein, gelatin, agar, diglycerin, glycerin, propylene glycol, polyethylene glycol, Vaseline, paraffin, stearyl alcohol, stearic acid, human serum albumin (HSA), mannitol, sorbitol, lactose, a pharmaceutically acceptable surfactant and the like. Additives used are chosen from, but not limited to, the above or combinations thereof, as appropriate, depending on the dosage form.

Formulation of the pharmaceutical composition will vary according to the route of administration selected (e.g., solution, emulsion). An appropriate composition comprising the antibody to be administered can be prepared in a physiologically acceptable vehicle or carrier. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. Intravenous vehicles can include various additives, preservatives, or fluid, nutrient or electrolyte replenishers

A variety of aqueous carriers, e.g., water, buffered water, 0.4% saline, 0.3% glycine, or aqueous suspensions may contain the active compound(s) in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally occurring phosphatide, for example lecithin, or condensation products of an alkylene oxide with fatty acids, for example polyoxyethylene stearate, or condensation products of ethylene oxide with long chain aliphatic alcohols, for example heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate.

The MSC, or MSC and allergen, composition can be lyophilized for storage and reconstituted in a suitable carrier prior to use. Any suitable lyophilization and reconstitution techniques can be employed. It will be appreciated by those skilled in the art that lyophilization and reconstitution can lead to varying degrees of MSC loss and/or allergen activity loss and that use levels may have to be adjusted to compensate.

Dispersible powders and granules suitable for preparation of an aqueous suspension by the addition of water provide the active cells and/or allergen in admixture with a dispersing or wetting agent, suspending agent and one or more preservatives. Suitable dispersing or wetting agents and suspending agents are exemplified by those already mentioned above.

The pharmaceutical compositions may be in the form of a sterile injectable aqueous, oleaginous suspension, dispersion or sterile powder for the extemporaneous preparation of sterile injectable solutions or dispersions. The suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, for example as a solution in 1,3-butanediol. The carrier can be a solvent or dispersion medium containing, for example, water, ethanol, polyol (for example, glycerol, propylene glycol, and liquid polyethylene glycol), suitable mixtures thereof, vegetable oils, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

In all cases the form must be sterile and must be fluid to the extent that acceptable syringability exists. The proper fluidity can be maintained, for example, by the use of a coating, such as lecithin, by the maintenance of the required particle size in the case of dispersion and by the use of surfactants. It must be stable under the conditions of manufacture and storage and must be preserved against the contaminating action of microorganisms, such as bacteria and fungi. The prevention of the action of microorganisms can be brought about by various antibacterial an antifungal agents, for example, parabens, chlorobutanol, phenol, sorbic acid, thimerosal, provided that such agents do not deleteriously affect MSCs to an unacceptable extent in compositions comprising such MSCs. In many cases, it will be desirable to include isotonic agents, for example, sugars or sodium chloride. Prolonged absorption of the injectable compositions can be brought about by the use in the compositions of agents delaying absorption, for example, aluminum monostearate and gelatin.

Compositions useful for administration may be formulated with uptake or absorption enhancers to increase their efficacy. Such enhancer include for example, salicylate, glycocholate/linoleate, glycholate, aprotinin, bacitracin, SDS, caprate and the like. See, e.g., Fix (J. Pharm. Sci., 85:1282-1285, 1996) and Oliyai and Stella (Ann. Rev. Pharmacol. Toxicol., 32:521-544, 1993).

In addition, the properties of hydrophilicity and hydrophobicity of the compositions contemplated for use in the methods of the disclosure are well balanced, thereby enhancing their utility for both in vitro and especially in vivo uses, while other compositions lacking such balance are of substantially less utility. Specifically, compositions contemplated for use in the methods disclosed herein have an appropriate degree of solubility in aqueous media, which permits absorption and bioavailability in the body, while also having a degree of solubility in lipids that permits the compounds to traverse the cell membrane to a putative site of action. Thus, MSC compositions contemplated are maximally effective when they can be delivered to the target site.

In one aspect, methods of the disclosure include a step of administration of a pharmaceutical composition. Methods of the disclosure are performed using any medically accepted means for introducing a therapeutic directly or indirectly into a mammalian subject, including but not limited to injections, oral ingestion, intranasal, topical, transdermal, parenteral, inhalation spray, vaginal, or rectal administration, as noted above. The term parenteral as used herein includes subcutaneous, intravenous, intramuscular, and intracisternal injections, as well as catheter or infusion techniques. Administration by, intradermal, intramammary, intraperitoneal, intrathecal, retrobulbar, intrapulmonary injection and or surgical implantation at a particular site is contemplated as well.

In one embodiment, administration is performed by direct injection or via a sustained delivery or sustained release mechanism, which can deliver the formulation internally. For example, biodegradable microspheres or capsules or other biodegradable polymer configurations capable of sustained delivery of a composition (e.g., a soluble polypeptide, antibody, or small molecule) can be included in the formulations of the disclosure.

Therapeutic compositions may also be delivered to the patient at multiple sites. The multiple administrations may be rendered simultaneously or may be administered over a continuous period of time.

In the working examples provided below, MSCs were studied in experimental AR mouse models and the results indicated that MSC migration through nasal mucosa inhibited eosinophil-mediated inflammation. Given the capacity of MSCs to also enhance some immune responses, the effects of MSCs on the proliferation of peripheral blood mononuclear cells (PBMCs) in allergic (sensitive to dust mite allergen Der p I (DM)), in allergic asthma (AA) subjects, and in patients with AR (AR patients do not have asthma) was examined. This study investigated a role for MSCs in antigen-challenged peripheral blood mononuclear cells (PBMCs) from patients with allergic rhinitis. Disclosed herein is an enhancing immune effect of MSCs to pollen provocation in allergic rhinitis. The enhancement was caused by MSCs reacting to the antigen and presenting to CD4⁺ T-cells.

Proliferation was studied by tritiated thymidine uptake, with or without MSCs. In the case of allergic asthma, the refractory response of PBMCs to DM was examined after preconditioned with MSCs and by repeated challenge with low-dose DM. Flow cytometry was used to evaluate regulatory T-cells (T_{reg}) and dendritic cells (DC). ELISA was used to evaluate cytokine production.

In a study of allergic asthma, seven subjects met the inclusion/exclusion criteria. This study disclosed the suppressive effect of MSCs on DM-activated PBMCs in allergic asthma, but not in DM-sensitive/non-asthmatic subjects. The suppressive effect could not be explained by an increase in CD4/CD25/FoxP3⁺ cells, which is consistent for regulatory T cells (T_{regs}), but MSC and DM-challenged PBMCs resulted in decreased interferon γ (IFNγ) and concomitant increase in interleukin 10 (IL-10). MSCs and DM caused a decrease in mature dendritic cells (DCs) in allergic asthma subjects. The suppressive effect of MSCs was specific for DM because MSCs did not suppress challenges to Tetanus toxoid. The experimental evidence indicated that MSCs are involved in resetting the immune response to make the cells more refractory to challenge with DM antigen. Accordingly, MSCs are useful in attenuating inflammatory responses to allergic triggers in asthma patients, and “off-the-shelf” MSCs are used for this purpose.

MSCs from a variety of sources are used to attenuate the immune response of peripheral blood mononuclear cells (PBMCs) to antigenic stimulation, particularly PBMCs from individuals with allergic asthma. We found that MSCs suppressed proliferation of PBMCs from allergic asthma patients in response to challenge with dust mite antigen. Also, small challenges of MSC-preconditioned PBMCs resulted in PBMCs that were refractory to further stimulation with dust mite antigen. In contrast, the addition of MSCs to stimulated PBMCs from allergic rhinitis worsened the outcome. This was due to the immune-enhancing role of MSCs, specifically, antigen presentation.

According to the disclosure, MSCs are administered to a cell during or after exposure to an antigen to suppress the response of the cell to the antigen. The cell is preferably a peripheral blood mononuclear cell and more preferably a peripheral blood mononuclear cell from a subject with allergic asthma. The antigen used in a working example disclosed herein was dust mite allergen (Der p I). In the case of subjects having allergic asthma, exposure of such subjects to MSCs is expected to have an analogous effect, or response, when such subjects are exposed to an allergen capable of inducing asthma in that subject. Specifically, not but exclusively, these allergens may encompass dust mite allergen (Der p I), ragweed, pollen, or rye grass.

In another embodiment, MSCs are used to precondition a cell and thereby render it refractory to challenge with an antigen. The cell is a peripheral blood mononuclear cell, e.g., a peripheral blood mononuclear cell from a subject with allergic asthma.

In another embodiment, MSCs are administered to a cell in combination with low doses of an antigen to render it refractory to challenge with an antigen. An exemplary cell is a peripheral blood mononuclear cell such as a peripheral blood mononuclear cell from a subject with allergic asthma.

The disclosure will be more fully understood by reference to the following examples which detail exemplary embodiments of the disclosure. The examples should not, however, be construed as limiting the scope of the disclosure.

### Example 1

### Methods

### Reagents

Phosphate Buffered Saline (PBS), Ficoll Hypaque, RPMI1640 and DMEM were purchased from Sigma, St Louis (MO). Fetal Calf Serum (FCS) was purchased from Hyclone Laboratories (Logan, Utah). DM antigen was obtained from Hollister-Stier (Spokane, Wash.). The solution contains Dermatophagoides farinae 15,000 AU/ml and Dermatophagoides pteronyssinus 15,000 AU/ml. T. toxoid was obtained from Squibb and distributed by Antigen Supply House (Northridge, Calif.). Allergen extracts were purchased from Hollister-Stier Laboratories (Spokane, Wash.).

### Study Subjects

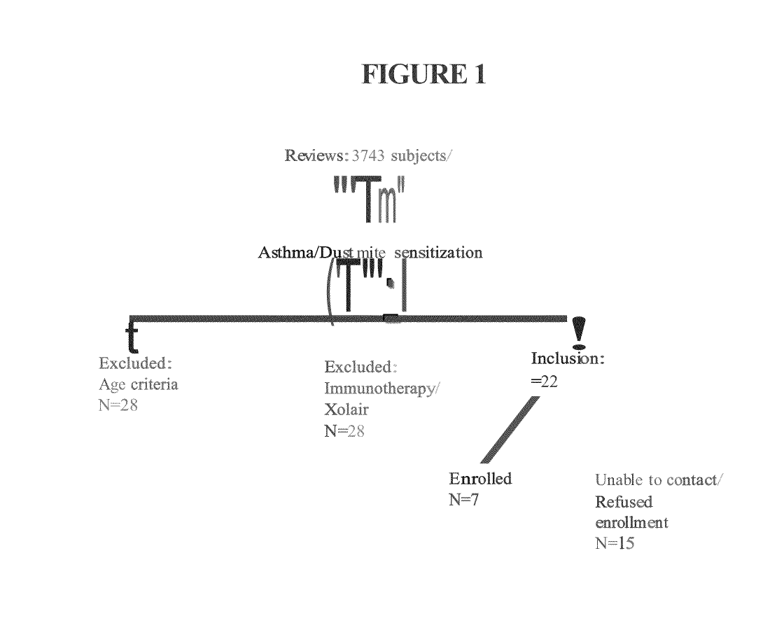

Subjects were recruited from the Allergy and Immunology clinic, New Jersey Medical School (Newark, N.J.) and voluntarily donated 10 mL of blood. The subjects were retrospectively reviewed for the last 3 yrs (FIG. 1), using the diagnoses code for allergic asthma and allergic rhinitis. In the case of allergic asthma, inclusion criteria included ages, 18 to 40 yrs and DM sensitivity, determined by history and by skin prick testing. Exclusion criteria include the following co-morbid conditions: heart disease, diabetes, cancer, pregnancy, HIV, immunodeficiency, and auto-immune disease; use of immune modulator; use of systemic steroids within 3 months prior of the blood draw or asthma flare within 1 month prior to blood draw. As shown in FIG. 1, among the 3743 screened subjects, 78 met the criteria for allergic asthma and DM sensitivity. Of these, 28 cases were not within the acceptable age range; 28 cases were excluded due to requirements for immune therapy and treatment with Xolair. Among the remaining 22 subjects who met the inclusion criteria, 15 either could not be contacted or refused enrollment into the study. Finally, seven subjects were recruited in the allergic asthma group. Most of the subjects were allergic to more than one antigen, but two were monosensitized to DM. Four healthy controls, with negative positive skin test for a panel of allergens and four DM-sensitive non-asthmatic controls volunteered for the study. All control subjects were evaluated for history of allergy, had physical examinations and were given allergy skin tests.

Study subjects for AR, ages 18 to 40 years, with known allergy to grass or ragweed allergy were enrolled between August, 2010 and June, 2012. The patient's physician diagnosed AR, based on the following clinical symptoms: nasal congestion, rhinorrhea, sneezing, nasal itching and itchy, watery eyes and skin reactivity to pollen. A positive response on skin prick testing to pollen was defined as a wheal response of >3 mm greater as compared to the negative saline control. Subjects were included if skin prick testing was done within one year of enrollment. In addition, patients were enrolled if they were off antihistamine for at least five days. Subjects with significant co-morbid conditions, such as heart disease, atopic dermatitis, immunodeficiency, diabetes, cancer, HIV and pregnancy were excluded. Patients were also excluded if they were receiving immunotherapy or if they were on oral corticosteroids within 6 months of the date of study. Nasal corticosteroid therapy was allowed. Informed consent was obtained from all study subjects.

The demographics and allergic state of patients are shown in Table 1. Blood samples were drawn at a time when most subjects were asymptomatic or slightly symptomatic and were considered healthy. None of the patient has asthma except for Subject 6, who was included as a control for suppression by MSCs².

The Institutional Review Board of University of Medicine and Dentistry of New Jersey, Newark Campus approved the study protocol. We obtained informed consent from all subjects enrolled in the study.

### Isolation of Peripheral Blood Mononuclear Cells (PBMCs)

Approximately 10 mL of blood was obtained from study subjects in heparinized tubes. The PBMCs were immediately isolated by Ficoll-Hypaque gradient separation. The blood was diluted with equal volume of sterile PBS and then added to an equal volume of Histopaque. The buffy coat containing PBMCs was collected and then washed three times in PBS. After the final wash, PBMCs were resuspended at 10⁶/mL RPMI 1640 with 10% FCS.

### Mesenchymal Stem Cell Culture (MSCs)

Bone marrow aspirates were obtained from the posterior iliac crest of healthy donors, ages between 18 and 30 years. Use of human subjects was adherent to the guidelines approved by the Institutional Review Board of the University of Medicine and Dentistry of New Jersey. MSCs were cultured from bone marrow aspirates as previously described^{3,4}. Unfractionated aspirates were added to DMEM with 10% FCS (D10 medium) and transferred to plasma-treated, tissue culture Falcon 3003 petri dishes (Fischer Scientific). Plates were incubated and at day 3, the red blood cells and granulocytes were removed by Ficoll Hypaque density gradient. The mononuclear fraction was replaced in the dishes with fresh D10 medium. Fifty percent of the media were replaced weekly until the adherent cells were about 80% confluent. At this time, the adherent cells were serially passaged at least four times. At this time, the cells were symmetric, CD14⁻, CD29⁺, CD44⁺, CD45⁻, CD105⁺, prolyl-4-hydroxylase⁻.

### Proliferation

DM Stimulation:

Mononuclear cells (PBMCs) were isolated from blood by Ficoll Hypaque gradient. PBMCs were studied in proliferation assays by ³H-TdR incorporation. PBMCs (2×10⁵) were placed in 96-well flat bottomed plates in 200 μL of RPMI 1640 containing 10% FCS. The addition of FCS was necessary to maintain viability after 3 days. Wells were stimulated with dust mite antigen (5 μg/mL DM) or tetanus toxoid (1/100 final dilution)^{1,3} or unstimulated. Dose-response and time course studies indicated 5 g as the optimum concentration. In parallel, MSCs (2×10³) were added into similar stimulated and unstimulated cultures. At day 4, each well was pulsed with 1 μCi/mL ³H-TdR. On day 5, the cells were harvested with a PhD cell harvester (Cambridge Technologies, Cambridge, Mass.) onto glass-fiber filters (Cambridge Technologies). Radioactive incorporation was acquired on a liquid scintillation counter (Beckman; Fullerton, Calif.). The stimulation indices were calculated as disintegration per min (dpm) of experimental points/dpm of unstimulated PBMCs.

Others:

The proliferation assay was performed as above using stimulator allergen based on the subject's known allergies (Table 1). Pollen (Hollister-Stier) was added at 5 μL/mL using the following: standardized Rye Grass (Lot # E08L1341); Timothy Grass (Lot #F08L1362); Bermuda (Lot #G09L7298); Meadow Fescue (Lot #C08J0534); short Ragweed (Lot #E10F0143). Except for Ragweed, the stock concentrations of all allergens were 10,000 BAU/mL. The stock concentration of Ragweed was 1:20 weight/volume with an Amba1 content of 191. The optimal concentrations for each allergen were determined in titration assays for each subject in dose-response curves. The concentrations, at peak proliferation, were selected for all studies.

### Pre-Conditioned PBMCs

The proliferation studies above were repeated, but with PBMCs, pre-incubated (preconditioned) with MSCs. In three independent studies, MSCs were pre-incubated with PBMCs from patients with: (a) allergic asthma; or (b) DM sensitivity without allergic asthma. On day 5, the MSC-free PBMCs were analyzed for responses to optimal DM (5 μg/mL) in proliferation assays, as described above.

### Repeated Exposure of PBMCs to Dust Mite and MSCs

PBMCs (10⁶/mL) were resuspended in RPMI1640 with 10% FBS and then exposed to repeated doses of DM as follows: 0.5 μg on Day 0, 0.25 μg on Day 3, 0.25 μg on Day 5, 0.125 μg on Day 7 and 0.125 μg on Day 10. MSCs (10⁵) were added on Days 0, 5, and 10 to the flasks. In parallel, flasks contained optimal (5 μg/mL) DM or media alone. On Day 12, the PBMCs were thoroughly washed and then studied in proliferation assay in the absence of MSCs, as described above.

### Flow Cytometry for T_{Regs} and DCs/Cytokine Quantitation

Flow cytometry for T_{regs} was performed as previously described⁴, using the Human Regulatory T Cell Staining Kit from e-Bioscience (San Diego, Calif.). The kit included FITC-anti-CD4, APC-anti-CD25 and PE-anti-FoxP3. DC analyses were performed by 4-color flow cytometry using V450-CD86 (Becton Dickinson), PE-CD14 (Becton Dickinson), APC-HLA-DR (Caltag Laboratories, Burlingame, Calif.) and FITC-anti-CD64 (Becton Dickinson). After labeling, cells were analyzed with the LSRII system (BD Biosciences). Parallel studies contained isotype controls, all from Becton Dickinson. Positive control BD Compbeads (BD Bioscience) were labeled with the all fluorochrome-tagged antibodies. The labeled cells were analyzed with the FACSCalibur system (BD Biosciences, San Jose, Calif.).

The media from PBMCs cultured alone or stimulated with DM and/or MSCs after 5 days were quantitated for IFNγ and IL-10 at Cytokine Core Lab, University of Maryland, Baltimore, Md.

### Immunoprecipitation/Western Blot

Immunoprecipitation of Class II, major histocompatibility complex, transactivator (CIITA) was performed with cytoplasmic and nuclear cell extracts using the Protein G-Agrose kit (Roche Applied Bioscience, Indianapolis, Ind.). Briefly, the extracts were incubated with anti-CIITA (Santa Cruz, Santa Cruz, Calif.) at 1/500 final dilution at 4° C. over night. After this, the reactions were incubated with protein-G agarose (1/50) at 4° C. for 4 h on a rocking platform. The reactions then were centrifuged at 4° C., 12000×g for 15 min, and the pellets were washed once with 1×PBS and resuspended in 1× sample buffer containing 0.5% β-ME and then analyzed by western blots with 20 μg of total protein.

The samples were electrophoresed on a 12% Mini-PROTEAN Precast Gel (Bio-RAD, Hercules, Calif.) and then transferred to polyvinylidene difluoride membranes (PerkinElmer, Whaltham, Mass.). The membranes were incubated with anti-CIITA at 1/500 dilution, 4° C. overnight; washed; incubated with HRP-conjugated goat anti-mouse IgG (1/1000) for 2 h at 4° C. HRP was developed with chemiluminescence detection reagents (Thermo Scientific). The molecular weight was determined with SeeBlue^{M} plus 2 Pre-stained standards (Life Technology).

Western blot for HLA-DR used rabbit polyclonal anti-HLA-DRa; ribosomal protein used goat polyclonal antibody; Acetyl-Histone H3 used rabbit polyclonal IgG. All antibodies were purchased from (Lake Placid); β-actin used murine monoclonal IgG (Sigma).

### CIITA Knockdown

CIITA shRNA were purchased from Origene (Rockville, Md.). Four different inserts were tested for the most efficient to knockdown CIITA. The inserts were placed in pRFP-C-RS plasmid under U6 promoter: FI355589, TCA GGC AGC AGA GGA GAA GTT CAC CAT CG (SEQ ID NO:1); FI355590, ACT GCG ACC AGT TCA GCA GGC TGT TGT GT (SEQ ID NO:2); FI355591, CCA GGC ATA CGT GAT GCG CTA CTT TGA GA (SEQ ID NO:3); FI355592, ACC TGA CCG CGT TCT GCT CAT CCT AGA CG (SEQ ID NO:4). The plasmid and vector alone were transfected in MSCs as described¹. Transfections were performed with the Effectene Transfection Reagent Kit (Qiagen). At 72 h and one week, MSCs were studied for CIITA by western blot. The results indicated efficient knockdown with the FI355592 plasmid.

### Antigen Presenting Cell (APC) Assay

Day 1, Cell Activation:

PBMCs (5×10⁶/ml) were incubated with optimum dose of ragweed (5 μL/ml). Unactivated cells contained only media. Day 4, Pulsing: MSCs (2×10⁴/ml) were incubated for 24 h with 2 μg/ml of Ragweed. Unpulsed MSCs omitted antigen. Day 5, Enrichment of CD4⁺ T-cells: CD4+ cells were enriched by negative selection of other immune cell subsets. PBMCs (10⁶/ml) were incubated with a cocktail of antibodies: CD3, CD8, CD11, CD56, CD20, each at 1/200 final dilution. After one h of incubation on ice, cells were washed with PBS and resuspended in 0.5 mL of PBS and 100 μL of Dynabead goat anti-mouse IgG (Invitrogen). The Dynabead-coupled cells were removed with a magnetic separator. The negative population was analyzed for CD4 by flow cytometry and the result indicated >90% labeling with anti-CD4. Day 5, Assay: Pulsed MSCs were resuspended in DMEM with 10% FCS at 10⁶/ml and then subjected to 2000 rads of γ-irradiation. Irradiation rendered the cells in cycling quiescence, but metabolically active. CD4⁺ enriched cells (4×10⁴/ml) were added to 50, 10², 10³ or 10⁴/ml to the γ-irradiated MSCs. After 24 h, cells were pulsed with 1 μCi of [methyl-³H]-TdR/well. After 16 h, cells were harvested, analyzed for radioactive incorporation and the simulation indices were calculated by dividing the dpm of experimental points by dpm of unactivated CD4⁺ T-cells.

### Statistical Analyses

Data were analyzed using analysis of variance and Tukey-Kramer multiple comparisons test. We considered a p value less than or equal to 0.05 as significant.

### Results

### Allergic Asthma

### Antigenic Stimulation of Dust Mite on Responsive and Non-Responsive PBMCs

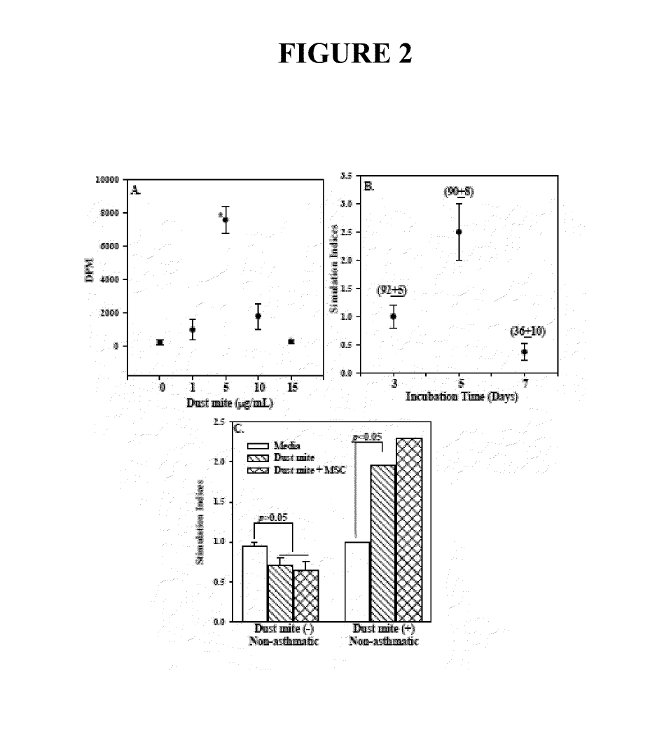

We first studied the specificity of dust mite antigen (DM) in proliferation of PBMCs. We compared PBMCs from four healthy controls with negative skin tests for dust mite to seven allergic asthmatic subjects, each studied in three independent experiments. A third group included in the studies comprised subjects who showed positive skin tests for dust mite and declared no history of asthma, and was designated as a dust mite sensitive (+)/non-asthmatic control. The stimulation indices (S.I.) of dust mite-stimulated PBMCs from subjects with negative skin test were comparable (p>0.05) to media control (FIG. 2C, left group), indicating insensitivity/lack of proliferation of the PBMCs to dust mite challenge. In contrast, the subjects with positive skin test showed significant (p<0.05) proliferation as compared to media control (FIG. 2C, right group). The addition of MSCs did not show significant (p>0.05) effect on the proliferation of PBMCs (FIG. 2C, right group, diagonal vs. hatched bars). In summary, this section shows the specificity of dust mite in the proliferation of PBMCs.

Effects of MSCs on the Proliferation of DM-Challenged PBMCs from Allergic Asthma

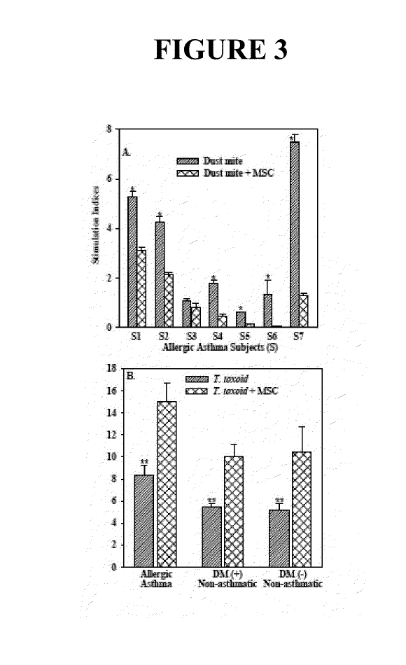

The studies described in FIG. 2 were repeated with the dust mite allergic asthma group with each subject studied in three independent experiments. In all subjects DM induced the proliferation of PBMCs (FIG. 3A, diagonal bars). MSCs significantly (p<0.05) reduced the proliferation for all subjects except subject 3 (S3) (FIG. 3A, hatched bar). The decreases for S1 and S2 were 1.6 and 1.7 fold, respectively. These suppressions were significantly (p<0.05) less than the other subjects with S6 showing a 19-fold decrease. In summary, MSCs significantly (p<0.01) decreased the proliferation of PBMCs in response to DM among six of the seven allergic asthma subjects. Our subjects all received boost of T. toxoid. To determine if the effect on MSCs is antigen specific rather than allergen specific, we repeated the above studies with T. toxoid. In all cases proliferation was enhanced further with MSCs (FIG. 3B). In summary, this shows the suppression by MSCs of PBMCs from allergic asthma subjects was specific for DM.

T_{Regs} and DC in DM-Challenged PBMCs from Allergic Asthma Subjects

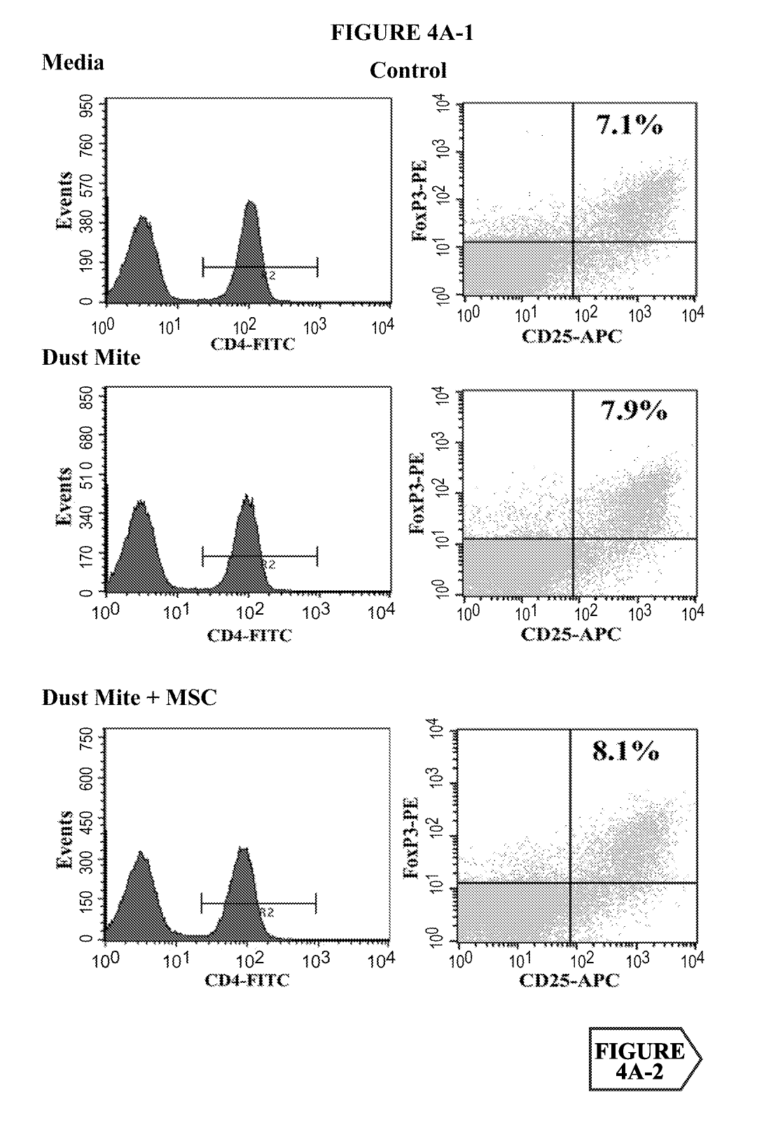

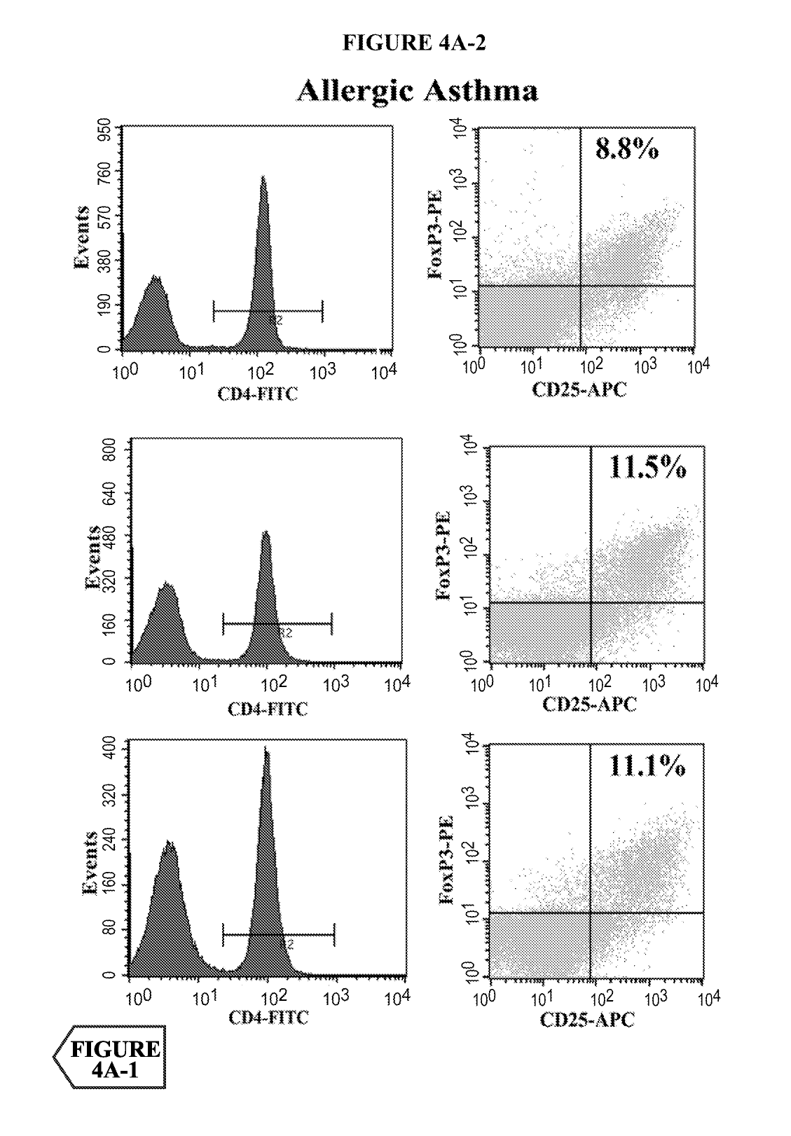

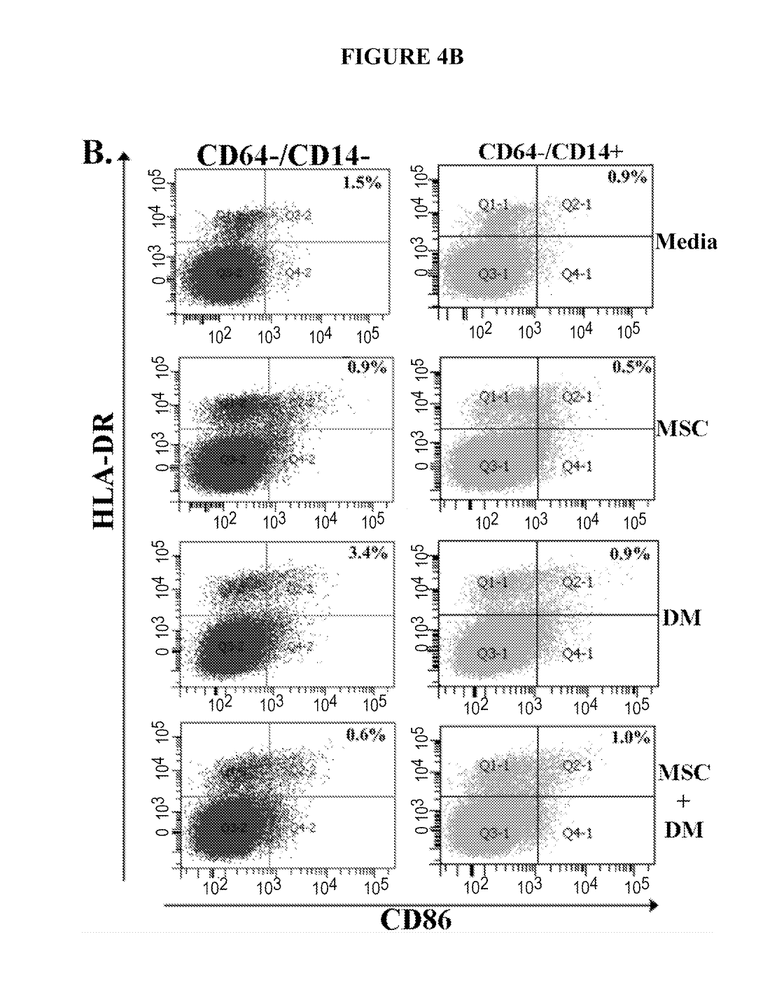

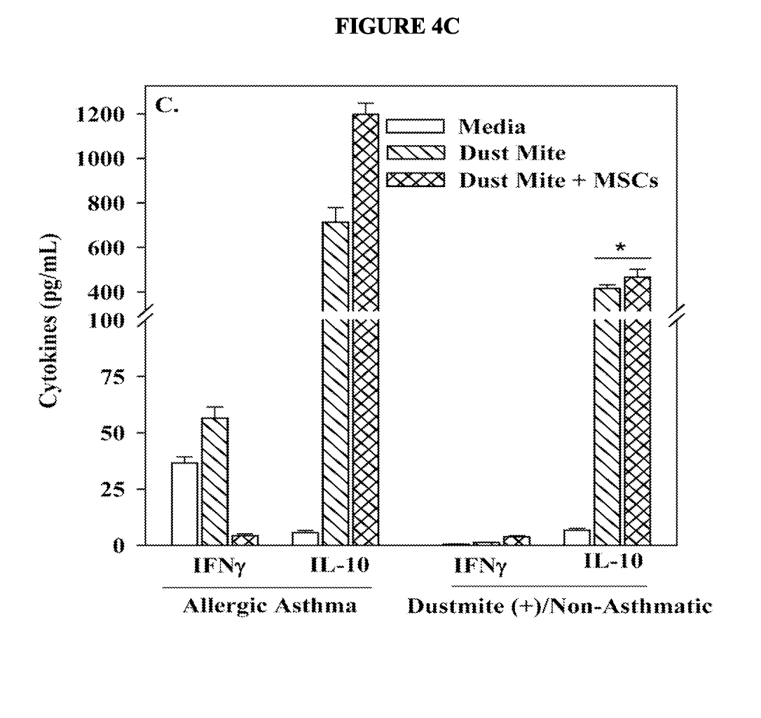

MSCs have been reported to cause immune suppression through secondary induction of T_{regs}. We therefore challenged PBMCs for 5 days in the presence or absence of MSCs and assayed for T_{regs}. Flow cytometry for CD4, Foxp3 and CD25 revealed no significant (p>0.05) change in T_{reg} (FIG. 4A), although there appeared a trend for an increase in T_{reg} in the allergic asthma subjects as compared to DM(−) non-asthmatic control (left panels). Since Foxp3 and CD25 do not always prove the presence or absence of T_{regs}, we quantitated the media for IFNγ and IL-10. The results showed significant increases in IL-10 but not IFNγ for both allergic asthma and allergic, non-asthmatic (FIG. 4C). The IL-10 levels in the non-asthmatic were significantly (p<0.05) less than similar conditions from the asthmatics. Also, IFNγ remained low in the non-asthmatic, despite stimulation with DM. In summary, the results showed no significant induction of T_{reg}, based on phenotype in the challenged AA subjects, regardless of MSCs but showed an increase in IL-10 levels, suggesting another mechanism of suppression.

Since it is unclear if T_{regs} are involved in the suppressive effects of MSCs (FIG. 4A), we studied the cultures for mature DCs. MSCs have been shown to inhibit monocyte-derived differentiation of DCs. Using the same experimental condition as for T_{reg} analyses, we performed multi-color flow cytometry for mature DC. We gated the CD64(−) fraction to eliminate granulocytes, and then performed further analyses, based on the expressions of CD14, HLA-DR and the mature co-stimulatory molecule CD86. In DM-treated PBMCs, there was about 6-fold increase in CD14−/CD86+ cells whereas no change was observed for the CD 14+ fraction (FIG. 4B), indicating that MSCs blunted DM-induced maturation of DCs.

### Non-Responsiveness to DM by MSC-Preconditioned PBMC

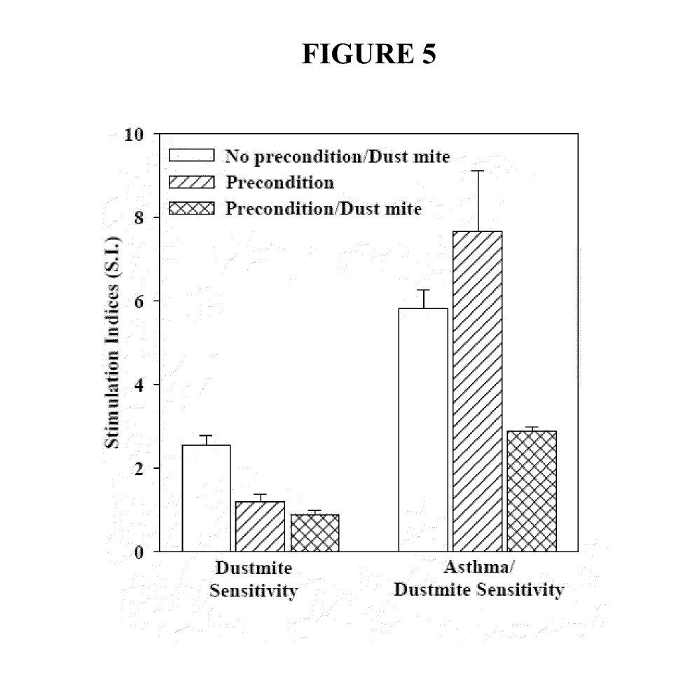

We evaluated the effect of preconditioning PBMCs with MSCs on their response to DM. PBMCs were pre-incubated with MSCs and at day 5, the MSC-free PBMCs were studied in proliferation assays with DM. As expected, the unconditioned PBMCs responded to DM (FIG. 5, open bars). Interestingly, the preconditioned PBMCs from allergic asthma subjects proliferated when studied alone (FIG. 5, middle bar of right group), but this was significantly (p<005) reduced when rechallenged with DM (FIG. 5, right group, hatched bar). PBMCs of DM sensitive/non-asthmatic subjects did not show similar refractory behavior to rechallenge with DM (FIG. 5, left group, hatched bar).

We next asked if preconditioning requires direct contact between PBMCs and MSCs. This was addressed in transwell cultures with MSCs in the inner wells and PBMCs in the outer wells, in the presence or absence of optimal (5 μg/mL) DM. After 5 days, the PBMCs were studied in proliferation assay, as above. PBMCs responded to DM with S.I., 6±0.8, indicating that preconditioning of PBMCs from allergic asthma requires direct contact with MSC.

Refractoriness of PBMCs by Repeated Challenge with Dust Mite Antigen and MSCs

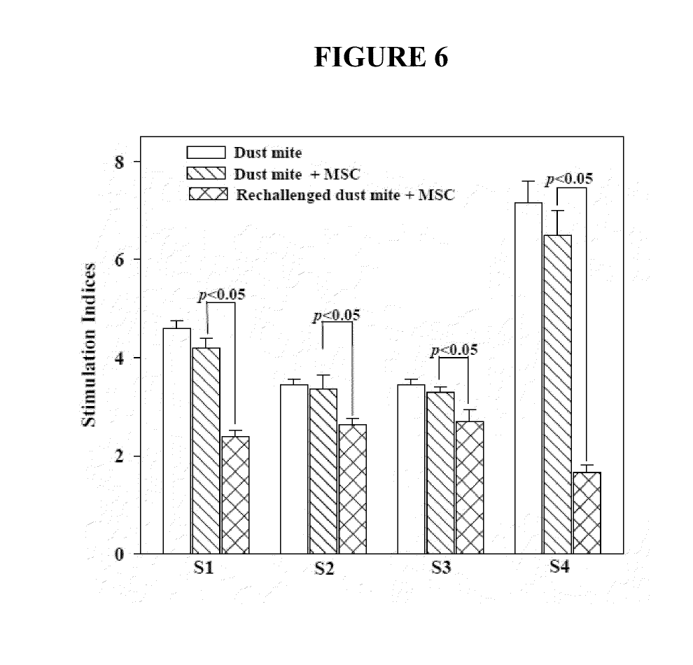

Refractoriness of the preconditioned PBMCs, together with a blunting effect on DC maturation (FIGS. 4B, 5) led us to ask if we can induce refractoriness with repeated low doses of DM, in the presence of MSCs. In four different subjects, each studied in three independent experiments, were rechallenged with optimal DM. Indeed, the S.I. of PBMCs were significantly decreased indicating refractoriness of the repeated challenge cells, similar to what would be expected of anergy (FIG. 6). The suppression could not be due to the FCS in the cultures, since parallel cultures with DM alone were able to respond to DM (FIG. 6, open bars).

### Results

### Allergic Rhinitis

PBMCs from subjects with rye grass and/or ragweed allergy who met the inclusion criteria were selected for the experimental study. A total of seven subjects were enrolled; one was excluded due to pre-existing anemia. Among the six subjects, two were sensitive to rye allergy and four with ragweed allergy. The control with allergic asthma was sensitive to both rye and ragweed allergies. Each analysis per subject was repeated in three different experiments, totaling six experimental points per allergen/subject.

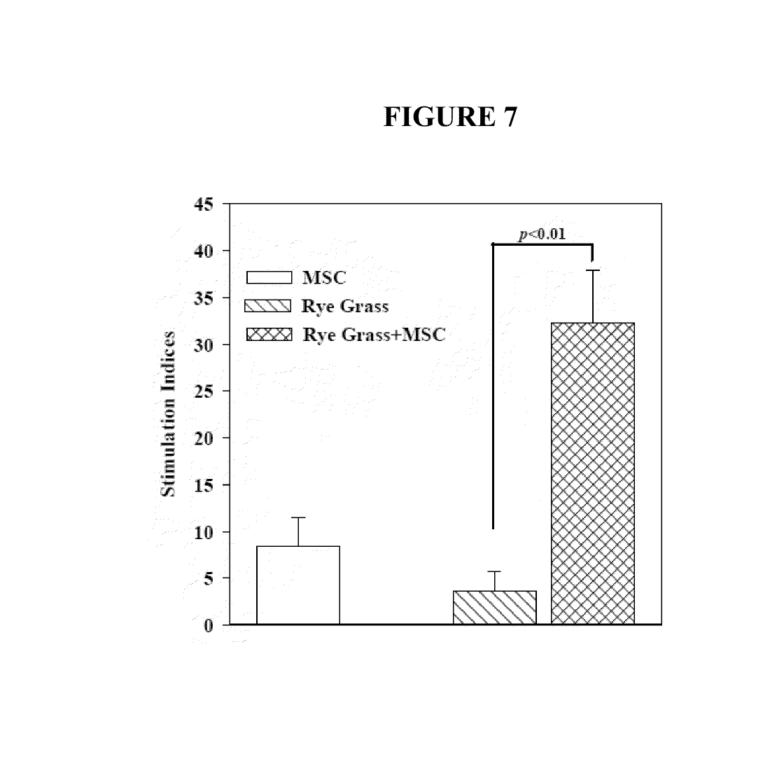

Low expression of MHC-II on MSCs can elicit an allogeneic response. In all of the studies, MSCs induced allogeneic stimulation on the PBMCs, as indicated in proliferation studies (FIGS. 7-9, open bars). Parallel studies with γ-irradiated MSCs (2,000 Rads) showed similar responses, indicating that the proliferation was from the PBMCs and not the MSCs.

MSCs Increased the Proliferation of PBMCs Challenged with Rye Grass

We first evaluated two different subjects, in three independent experiments with rye grass allergy. Dose-response studies with rye grass ranging between 1 and 20 μl/ml indicated optimum dose as 5 μl/ml. We and others previously showed allogeneic stimulation by MSCs on the proliferation of PBMCs. Therefore, as a control, PBMCs were challenged with allogeneic MSCs. As expected, the S.I. was 8.4±3 (±SD) (FIG. 7, open bar). PBMCs provoked with optimal rye grass showed S.I. of 3.7±2 (±SD), indicating significant (p<0.05), although low, response to rye grass (FIG. 7, diagonal bar). However, in the presence of MSCs, the proliferation was significantly (p<0.01) increased to S.I. of 32±5.7 (±SD) (FIG. 7, hatched bar). This was about 8-fold more than parallel cultures without MSCs but with rye grass (FIG. 7, diagonal bar). The increased proliferation by MSCs could not be explained by allogeneic stimulation alone, and suggested that MSCs may be acting as APCs. This set of experiments showed an enhancing effect of MSCs in rye-challenged PBMCs from patients with minimal to moderate baseline in vitro sensitivity to pollen challenge.

### Effects of MSCs on Ragweed-Challenged PBMCs

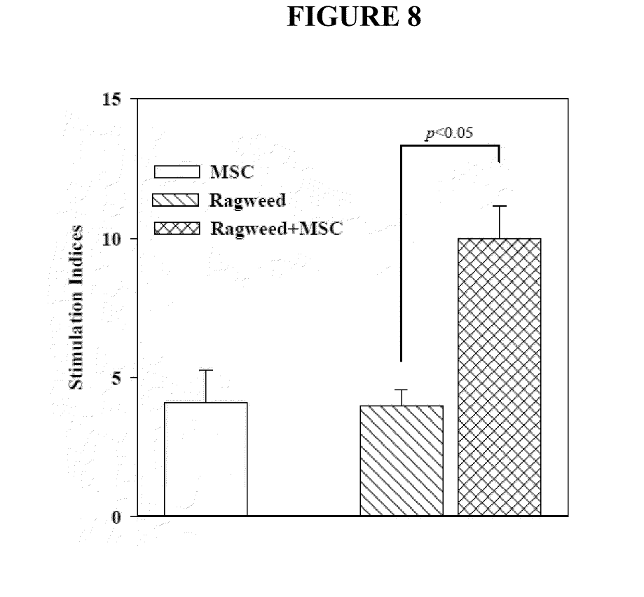

As MSCs have been shown to suppress inflammatory responses in the literature, the immune-enhancing effect of MSCs in rye-challenged cultures was unexpected. We therefore examined another allergen and selected ragweed. Four subjects were studied, each in three independent experiments. The baseline in vitro proliferative response to ragweed in two subjects was relatively modest in this experiment, but comparable to the response with rye grass (S.I. of 4±0.6, ±SD). The response to ragweed was similar to the allogeneic response by MSCs alone (FIG. 8 open bar vs. diagonal bar). However, MSCs and ragweed together, caused a significant (p<0.05) increase with an SI of about 10 (FIG. 8, diagonal bar vs. hatched bar). In summary, this section showed an enhancing effect of MSCs to ragweed-challenged PBMCs in six different experiments with known allergy to ragweed. There were modest in vitro responses to pollen at baseline.

### Effects of MSCs to Ragweed Allergen as a Function of In Vitro Sensitivity

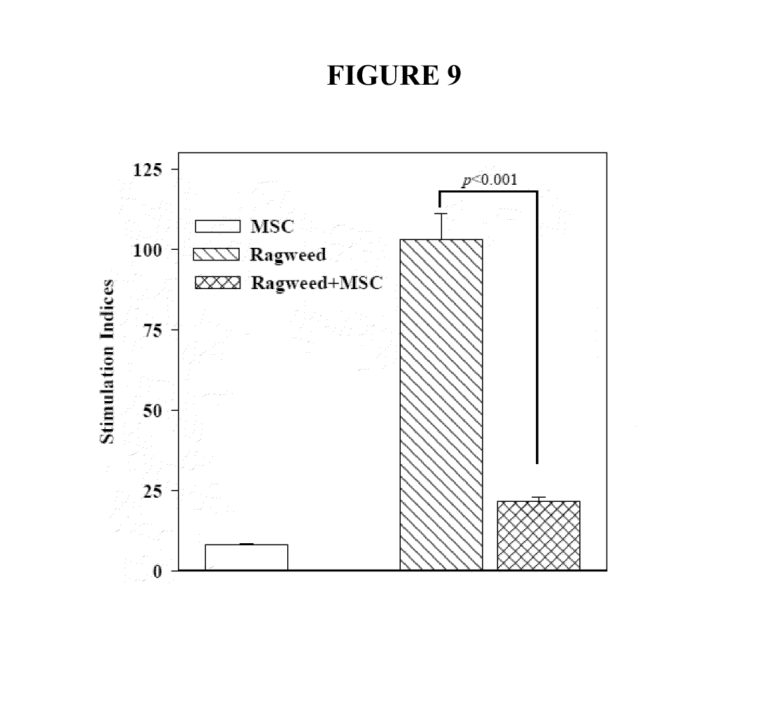

We previously reported on a suppressive effect of MSCs on allergic asthma using dust mite antigen. This was unlike the effect observed for allergic rhinitis without asthma (FIGS. 7 and 8). We therefore selected a subject (#6) with asthma and allergy to ragweed as an experimental control. We established proliferation studies as for FIGS. 7 and 8. In contrast to the other subjects, the subject with allergic asthma showed a relatively high sensitivity to ragweed with an S.I. of 103±8 (FIG. 9). As compared to the subjects with AR, the response to ragweed in the allergic asthma PBMCs was significantly greater than the allogeneic response with MSCs alone (FIG. 9, open bar versus diagonal bar). MSCs showed significant (p<0.001) reduction (4.8 fold decrease) in the proliferation of ragweed-challenged PBMCs from the allergic asthma subject (FIG. 9). This section showed observations that were consistent with a suppressive effect of MSCs on allergic asthma.

### Antigen Presenting Properties (APC) of Ragweed-Challenged MSCs

MSCs can function as APCs, including cross presentation via MHC-I. This could occur if the inflammatory response to the antigen is insufficient to license MSCs to become immune suppressor cells. We therefore investigated if the enhancing effect of MSCs could be explained by its APC property. PBMCs from ragweed-sensitive subjects were primed with the antigens for five days. The CD4(+) cells were then added to antigen-pulsed MSCs. Baseline control contained activated CD4(+) cells alone. Additional controls contained unpulsed MSCs added to activated and unactivated CD4+ cells.

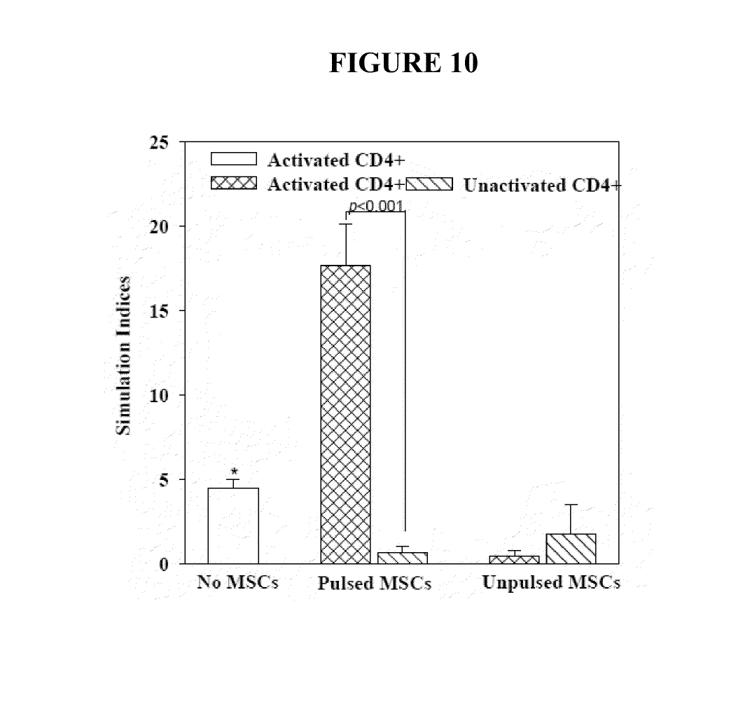

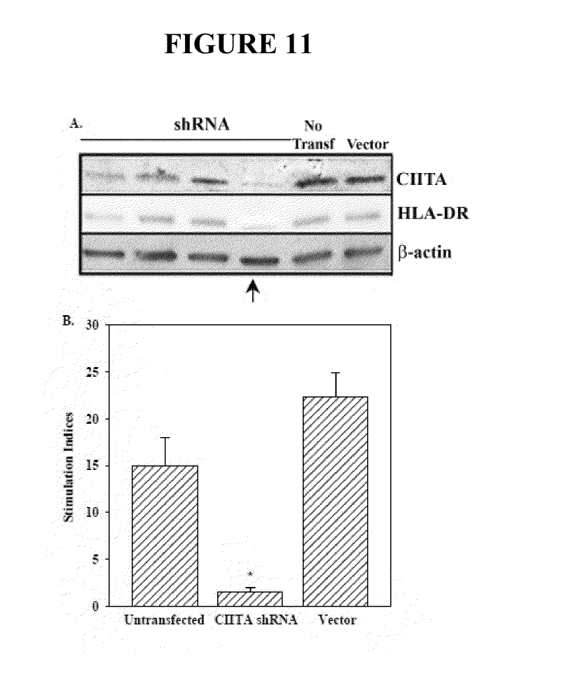

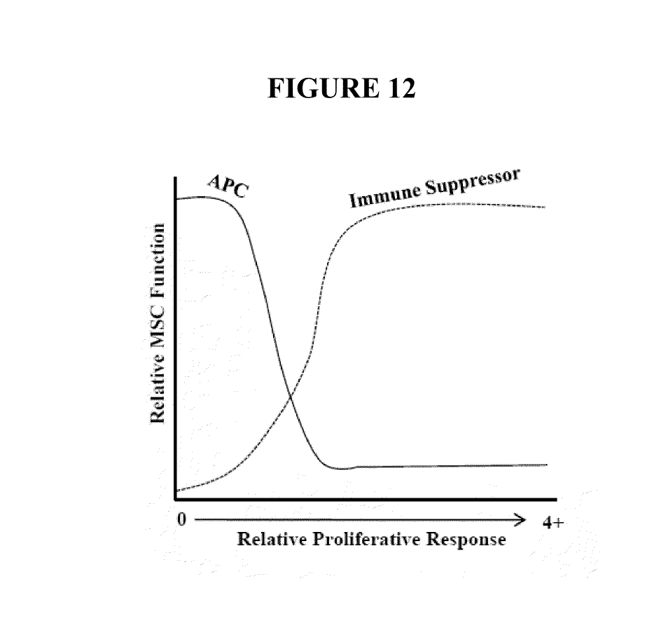

Ragweed-pulsed MSCs and pre-activated CD4(+) cells resulted in significant (p<0.01) proliferation as compared to unprimed CD4(+) cells and pulsed MSCs (FIG. 10, middle group). Interestingly, when unpulsed MSCs were added to activated CD4+ cells, the proliferation was significantly decreased (p<0.05) as compared to activated CD4+ cells alone (FIG. 11, Right group/hatched bar vs. open bar). This suggested that MSCs can exert immune suppressive functions when they are placed with highly activated CD4+ cells, which is consistent with the immune suppressive functions of MSCs. The proliferative effects were not due to non-specific outcome since the addition of unactivated CD4(+) cells and unpulsed MSCs resulted in baseline proliferation (FIG. 11/middle diagonal bar). In summary, the results indicated APC function of MSCs with ragweed, but also indicated immune suppressor functions of unpulsed MSCs to activated CD4+ cells.

### Blunted APC Function in CIITA Knockdown MSCs

MSCs can express low level of MHC-II, which provided the cells with the ability to exert APC functions. Since CIITA is a master regulator of MHC-II transcription, we studied the specificity of the APC functions by repeating the studies shown in FIG. 10, except with CIITA knockdown MSCs. We first tested four different shRNA constructs to identify the most efficient in knockdown of CIITA. Clone FI355592, shown with an arrow was determined to be most efficient and was therefore used in subsequent studies (FIG. 11A). The APC assay was performed with ragweed with MSCs, knockdown for CIITA, untransfected or vector-transfected. The results showed significant (p<0.01) reduction in APC by the CIITA knockdown MSCs. Overall, these studies showed the specificity of the APC function of MSCs to ragweed and indicated the involvement of MHC-II.

### Discussion

### Allergic Asthma

The literature has reported on dual immune functions of MSCs, namely, immune suppression and enhancement. Each type of response appears to occur during a narrow window, depending on the milieu. In this study, we observed a veto-like response since MSCs showed preference by suppressing PBMCs from allergic asthma subjects that are expected to be hyperactivated as compared to subjects with only allergic responses, mono- and poly-sensitized (FIGS. 2C, 3B). The results are not antigen-specific, but for allergens, as denoted by studies with T. toxoid (FIG. 3B). In the presence of MSCs, T. toxoid showed enhanced proliferation (FIG. 3B). We expect that this can be explained by the antigen presenting properties of MSCs.

PBMCs from subjects with allergic asthma showed significantly more response to DM as compared to similar challenges of PBMCs from DM(+) non-asthmatic (FIGS. 2C and 3). This type of outcome mimics a veto-like response since it is expected that PBMCs from allergic asthma would be more sensitive to stimulation with an allergen. In contrast to the inhibitory effect of MSCs on the proliferation of PBMCs from allergic asthma subjects, MSCs could not suppress DM-activated PBMCs from non-asthmatic/DM sensitive subjects (FIG. 2C). These findings are consistent with other studies in which MSCs could not suppress T. toxoid-challenged PBMCs. In fact, the addition of MSCs to the non-asthmatic/dust mite (+) PBMCs showed a trend towards increased proliferation of the PBMCs (FIG. 2/right group/hatched bar). We explain this finding by other studies showing an antigen presenting role of MSCs when IFN-γ level is low. It is possible that the relatively low proliferative response to dust mite prevented high level of IFNγ for antigen presenting function.

The suppressive effect of MSCs on the proliferation of PBMCs from allergic asthma subjects recapitulates inflammation, which would cause decreases in cytokine production. These findings are relevant to the translation of MSCs for allergic asthma, in light of the deleterious secondary effects of cytokines in allergic asthma.

MSC-mediated immune suppression has been linked to increases in T_{regs} and related cytokines. In this study, the level of T_{regs} was unchanged with MSCs and DM (FIG. 4A). We correlated these findings with specific cytokines and found an increase in IL-10 with no change in IFNγ for the DM/non-asthmatic group (FIG. 4C). In other studies, T_{reg} induction has been shown to reduce airway inflammation, caused by DM. Since these studies were done in vivo, the mice can be examined for long periods. It is possible that our 5-day model might be inadequate to induce T_{regs}. It is difficult to retain the cultures for longer periods without a high rate of cell death. Previous studies were done only with DM, which caused reduced responses in non-asthmatic subjects, indicating differences in the two models. The inability of MSCs to induce T_{regs}, despite its suppressive effect, is an advantage for translation in allergic asthma. If MSCs induce T_{regs} this could cause untoward effects such as those linked to immune suppressor drugs. T_{regs} have been reported to suppress T-cell responses by absorbing the proinflammatory IL-2. Since this method could not occur in our model, the effect of MSCs is by other mechanisms. In our studies, we have observed a blunt effect of MSCs in the maturation of DCs (FIG. 4B).

The mechanism and consequence of reduced mature DC in the presence of DM and MSCs require further exploration (FIG. 4B). While previous studies overwhelmingly showed evidence of MSC-mediated suppression through impediment of DC maturation, this model is different since the blunting effect is within a milieu of an allergen. MSCs can down regulate the expression of activating receptors on natural killer cells.

This study also provides a novel method by which tolerance to allergen could be achieved. We showed refractoriness of PBMCs to rechallenged DM by two methods, preconditioning with MSCs and repeated exposure to low dose DM and MSCs (FIGS. 5 and 6).

The varied responses of dust mite-challenged PBMCs to MSCs could not be explained by the subjects' BMI, although the answer to this question would benefit from a larger cohort. This question is pertinent since adipose tissues are a rich source of MSCs. If PBMCs from obese patients are exposed to adipose-derived MSCs, this might influence the allergic response and perhaps asthmatic effects. In this study, the grouping of subjects by BMI, i.e., 20-30 and 31-40, as a function of fold-decrease in PBMC proliferation showed no conclusive outcome.

In summary, this study showed a complex role for MSCs in allergic disease. The findings are significant to allergic asthma. Although dust mite was used as a model system, the findings can be extrapolated to other allergens.

### Discussion

### Allergic Rhinitis

We previously reported on an immune suppressive role for MSCs in the proliferation of PBMCs from patients with allergic asthma and the ability of MSCs to block the maturation of dendritic cells. We also showed evidence of immune tolerance with repeated exposure of MSCs and allergen to the lymphocytes. We began this study by assuming we will observe similar suppression with lymphocytes from subjects with AR but no asthma. However, we observed the opposite effects; showing an enhancing role of MSCs in lymphocyte proliferation (FIGS. 7 and 8). The data shown for ragweed and rye grass were similar for different allergens (not shown). MSCs exerted a suppressive effect on the proliferation of PBMCs from one subject (#6) with AR and asthma (FIG. 9). These findings were consistent with our other study with allergic asthma. The response of the subject with asthma and AR indicated that MSCs might be licensed to be immune suppressor cells when they are introduced within a milieu of high inflammation. Without wishing to be bound by theory, the response might be due to the subject's intrinsic sensitivity to allergen, the duration of allergen exposure and the underlying inflammation, in this case asthma. These findings suggest an immune-suppressive role for MSCs, particularly in an inflammatory environment.

The proliferation induced by MSCs were multiple-fold and beyond that expected from an allogeneic response to MSC. The presence of MSCs during exposure to antigen seemed to enhance the allergic response. Such a response may be due to MSCs acting as APCs, and presenting the pollen to lymphocytes. To test this hypothesis, we performed an APC assay with ragweed-allergic subjects. When activated PBMCs (primed with ragweed and in an inflammatory background) were exposed to MSC that were pulsed with ragweed, lymphocyte proliferation was markedly increased. This finding suggested that MSCs may in fact be presenting ragweed to the lymphocytes, stimulating such a dramatic response. The APC function can only occur if MHC-II is express and this requires the expression of the master regulator of transcription, CIITA. Its knockdown resulted in the loss of APC function (FIG. 11), supporting an APC function of MSCs in the presence of allergen.

While MSCs can present the ragweed to the lymphocytes, its addition to antigen-activated CD4+ cells indicated immune suppression (FIG. 10, right group/hatched bar). This suggested dual roles for MSCs with regards to immune activity. The addition of MSCs to activated PBMCs (primed with ragweed for several days) resulted in reduced PBMC proliferation. The addition of MSCs into an inflammatory microenvironment likely activated their immune suppressing capabilities. Although all the subjects showed 4+ sensitivity to ragweed on skin testing, this did not correlate with the degree of lymphocyte responsiveness seen in the assay and it is not possible to make clinical correlations with in vitro testing.

MSCs seem to have an immune suppressing role in AR. AR is an allergic disease characterized by an influx of eosinophils and Th2 cells, with the production of pro-inflammatory cytokines. Adipose tissue-derived stem cells inhibited eosinophilic inflammation in an AR mouse model by shifting from Th2 to Th1 response to allergens (decreased IL-4 and IL-5, and increased IFN-γ production). In other studies with a mouse model of asthma, the injection of MSCs at the time of ragweed challenge protected the mice from asthma-specific pathological changes. In these studies, the MSCs inhibited eosinophilic infiltration, excess mucus production, and showed a decrease in the production of Th2 cytokines.

Our in vitro findings, from human peripheral blood, indicated that the mechanism by which MSCs are licensed to be immunosuppressive might depend on the type of inflammatory disorders.

Surprisingly, antigen challenge in the presence of MSCs led to an exaggerated lymphoproliferative response. The clinical translation of such a finding is very important, and suggested that the subjects must be carefully selected. As the studies suggested, the infusion of MSCs in atopic individuals may worsen the symptoms and perhaps benefit. The type of response might depend on the timing of antigen exposure, the patient's in vitro sensitivity to the allergen and mostly if the patients are asthmatic. Given the clinical and economic consequences of poorly controlled AR, it is critical to understand how MSCs will affect a patient's allergic condition.

MSCs exhibited plasticity with regards to the immune response to pollen in patients with allergic rhinitis. The particular response of MSCs seemed to depend on the level of activation of PBMCs and in vitro sensitivity to allergen. In subjects with weak or moderate in vitro sensitivity to pollen, the addition of MSCs during pollen provocation may have immune enhancing effects by exerting antigen presenting properties. In subjects with baseline high in vitro sensitivity to pollen, or those pre-treated with pollen (creating a highly inflammatory microenvironment), however, MSCs may act as immune suppressor cells. In this study, the relative proliferative response to antigen might predict the immune effects of MSCs: antigen presentation versus suppressor function (FIG. 11).

TABLE 1
Baseline information on study subjects with allergic rhinitis

| | Age/ | Race/ | |
|---|---|---|---|
| Subject | Sex | Ethnicity | SPT* result |
| S1 | 30/ | Hispanic | Rye grass (2+), Positive grasses, |
| | Male | | trees, mold |
| S2 | 37/ | South | Rye grass (2+), Positive to trees, |
| | Male | Asian | dust mite |
| S3 | 20/ | Hispanic | Ragweed (4+), trees, cockroach, |
| | Male | | cat pelt |
| S4 | 23/ | African | Ragweed (4+) |
| | Female | American | |
| S5 | 27/ | Asian | Ragweed (4+), positive to Bermuda |
| | Female | | grass |
| S6 | 30/ | Hispanic | Asthma, Rye grass (4+), Ragweed |
| | Male | | (4+), positive to grasses and |
| | | | other weeds, dust mite, and cat |
| S7 | 30/ | Hispanic | Ragweed 3+ |
| | Female | | Positive to trees, grass (−) |

*SPT—Skin prick testing

### References for Example 1

1. Chan J L, Tang K C, Patel A P, Bonilla L M, Pierobon N, Ponzio N M et al. Antigen-presenting property of mesenchymal stem cells occurs during a narrow window at low levels of interferon-gamma. Blood 2006; 107(12):4817-24.
2. Kapoor S, Patel S A, Kartan S, Axelrod D, Capitle E, Rameshwar P. Tolerance-like mediated suppression by mesenchymal stem cells in patients with dust mite allergy-induced asthma. Journal of Allergy and Clinical Immunology 2012; 129(4):1094-101.
3. Potian J A, Aviv H, Ponzio N M, Harrison J S, Rameshwar P. Veto-Like Activity of Mesenchymal Stem Cells: Functional Discrimination Between Cellular Responses to Alloantigens and Recall Antigens. The Journal of Immunology 2003; 171(7):3426-34.
4. Patel S A, Meyer J R, Greco S J, Corcoran K E, Bryan M, Rameshwar P. Mesenchymal Stem Cells Protect Breast Cancer Cells through Regulatory T Cells: Role of Mesenchymal Stem Cell-Derived TGF-beta. The Journal of Immunology 2010; 184(10):5885-94.

### Example 2

Mesenchymal Stem Cells (MSCs) can suppress and/or enhance immune functions. MSCs show promise as a readily available cellular therapy for several disorders, including inflammation. In the experiments disclosed in this Example, the effects of MSCs on the proliferation of peripheral blood mononuclear cells (PBMCs) in allergic (dust mite, DM) and/or allergic asthma (AA) subjects were investigated. Proliferation was studied by tritiated thymidine uptake, with or without MSCs. The refractoriness of PBMCs to DM was examined after preconditioning with MSCs and by repeated challenge with low-dose DM. Flow cytometry was used to examine regulatory T-cells (T_{reg}) and dendritic cells (DC). ELISA was used to study cytokine production. Seven allergic asthma subjects met the inclusion/exclusion criteria. MSCs significantly (p<0.05) reduced the proliferation of six subjects with allergic asthma, but not allergy alone. The effect was specific to the allergen since MSCs did not affect challenges to Tetanus toxoid. There was no change in CD4/CD25/FoxP3⁺ cells, although IFNγ decreased and IL-10 increased. Mature DCs were increased by 6-fold. Refractoriness to DM was achieved by repeated exposure to low dose DM and MSCs, and PBMCs preconditioned with MSCs. MSCs suppressed the proliferation of DM-challenged PBMCs in allergic asthma subjects, but not from allergic subjects without asthma. MSCs blunted the maturation of DC but not T_{reg} cells. Repeated exposure to low dose DM and MSCs, as well as preconditioning of PBMCs with MSCs, caused refractoriness to DM. These findings have implications for the use of MSCs to attenuate the inflammatory responses to allergic triggers in asthma patients with ‘off the shelf’ MSCs.

In greater detail, a suppressive effect of MSCs on dust-mite- (DM-) activated PBMCs was seen in allergic asthma subjects, but not in DM-sensitive/non-asthmatic subjects. The suppressive effect could not be explained by an increase in CD4/CD25/FoxP3⁺ cells, which is consistent for regulatory T cells (T_{regs}), but MSC and DM-challenged PBMCs resulted in decreased IFNγ and concomitant increase in IL-10. MSCs and DM caused a decrease in mature dendritic cells (DCs) for allergic asthma subjects. The suppressive effect of MSCs was specific for DM because MSCs did not suppress challenges to Tetanus toxoid. The experimental evidence indicated that MSCs might be involved in resetting the immune response to make the cells more refractory to challenge with DM antigen, similar to other reports for MSCs on macrophages and sepsis¹⁵.

### Methods

### Reagents

Phosphate Buffered Saline (PBS), Ficoll Hypaque, RPMI1640 and DMEM were purchased from Sigma, St Louis (MO). FCS was purchased from Hyclone Laboratories (Logan, Utah). DM antigen was obtained from Hollister-Stier (Spokane, Wash.). The solution contains Dermatophagoides farinae 15,000 AU/ml and Dermatophagoides pteronyssinus 15,000 AU/ml. T. toxoid was obtained from Squibb and distributed by Antigen Supply House (Northridge, Calif.).

### Study Subjects

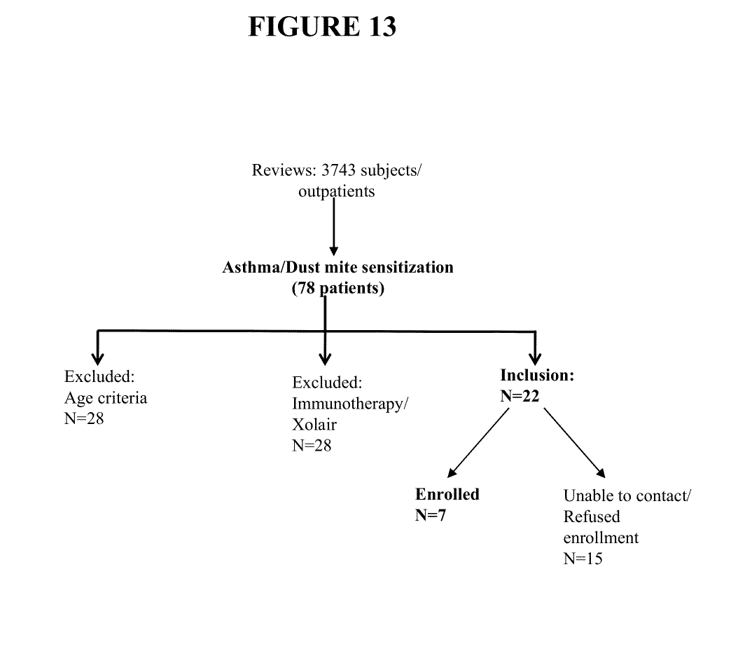

Subjects were recruited from the Allergy and Immunology clinic, New Jersey Medical School (Newark, N.J.) and voluntarily donated 10 mL of blood. The subjects were retrospectively reviewed for the last 3 yrs (FIG. 13), using the diagnoses code for allergic asthma. Inclusion criteria included ages, 18 to 40 yrs and DM sensitivity, determined by history and by skin prick testing. Exclusion criteria include the following co-morbid conditions: heart disease, diabetes, cancer, pregnancy, HIV, immunodeficiency, and auto-immune disease; use of immune modulator; use of systemic steroids within 3 months prior of the blood draw or asthma flare within the 1 month prior to blood draw.

As shown in FIG. 13, among the 3743 screened subjects, 78 met the criteria for allergic asthma and DM sensitivity. Of these, 28 cases were not within the acceptable age range; 28 cases were excluded due to requirements for immune therapy and treatment with Xolair. The remaining 22 subjects who met the inclusion criteria, 15 either could not be contacted or refused enrollment into the study. Finally, seven subjects were recruited in allergic asthma group. Most of the subjects were allergic to more than one antigen, but two were monosensitized to DM. Four healthy controls, with negative positive skin test for a panel of allergens and four DM-sensitive non-asthmatic controls volunteered for the study. All control subjects was evaluated for history of allergy, had physical examinations and, were given allergy skin tests. The Institutional Review Board of University of Medicine and Dentistry of New Jersey, Newark Campus approved the study protocol. We obtained informed consent from all subjects enrolled in the study.

### Mesenchymal Stem Cell Culture (MSCs)

Bone marrow aspirates were obtained from the posterior iliac crest of healthy donors, ages between 18 and 30 years. Use of human subjects was adherent to the guidelines approved by the Institutional Review Board of the University of Medicine and Dentistry of New Jersey. MSCs were cultured from bone marrow aspirates as previously described^{21,22}. Unfractionated aspirates were added to DMEM with 10% FCS (D10 medium) and transferred to plasma-treated, tissue culture Falcon 3003 petri dishes (Fischer Scientific). Plates were incubated and at day 3, the red blood cells and granulocytes were removed by Ficoll Hypaque density gradient. The mononuclear fraction was replaced in the dishes with fresh D10 medium. Fifty percent of the media were replaced weekly until the adherent cells were about 80% confluent. At this time, the adherent cells were serially passaged at least four times. At this time, the cells were symmetric, CD14⁻, CD29⁺, CD44⁺, CD45⁻, CD105⁺, prolyl-4-hydroxylase.

### Proliferation

Mononuclear cells (PBMCs) were isolated from blood by Ficoll Hypaque gradient. PBMCs were studied in proliferation assays by ³H-TdR incorporation. PBMCs (2×10⁵) were placed in 96-well flat bottomed plates in 200 μL of RPMI 1640 containing 10% FCS. The addition of FCS was necessary to maintain viability after 3 days. Wells were stimulated with dust mite antigen (5 μg/mL) or tetanus toxoid (1/100) final dilution^{21,23} or unstimulated. Dose-response and time course studies indicated 5 μg as optimum concentration. In parallel, MSCs (2×10³) were added into similar cultures, stimulated and unstimulated cultures. At day 4, each well was pulsed with 1 μCi/mL ³H-TdR. On day 5, the cells were harvested with a PhD cell harvester (Cambridge Technologies, Cambridge, Mass.) onto glass-fiber filters (Cambridge Technologies). Radioactive incorporation was acquired on a liquid scintillation counter (Beckman; Fullerton, Calif.). The stimulation indices were calculated as disintegration per min (dpm) of experimental points/dpm of unstimulated PBMCs.

### Pre-Conditioned PBMCs

The proliferation studies above were repeated, but with PBMCs, pre-incubated (preconditioned) with MSCs. PBMCs, in three independent studies, with cells from allergic asthma and only DM sensitivity were pre-incubated with MSCs. On day 5, the MSC-free PBMCs were analyzed for responses to optimal DM in proliferation assays, as described above.

### Repeated Exposure of PBMCs to Dust Mite and MSCs

PBMCs (10⁶/mL) were resuspended in RPMI1640 with 10% FBS and then exposed to repeated doses of DM as follows: 0.5 μg on Day 0, 0.25 μg on Day 3, 0.25 μg on Day 5, 0.125 μg on Day 7 and 0.125 μg on Day 10. MSCs (10⁵) were added on Days 0, 5, and 10 to the flasks. In parallel, flasks contained optimal DM or media alone. On Day 12, the PBMCs were thoroughly washed and then studied in proliferation assay in the absence of MSCs, as described above.

### Flow Cytometry for T_{Regs} and DCs/Cytokine Quantitation

Flow cytometry for T_{regs} was performed as previously described²², using the Human Regulatory T Cell Staining Kit from e-Bioscience (San Diego, Calif.). The kit included FITC-anti-CD4, APC-anti-CD25 and PE-anti-FoxP3. DC analyses were performed by 4-color flow cytometry using V450-CD86 (Becton Dickinson), PE-CD14 (Becton Dickinson), APC-HLA-DR (Caltag Laboratories, Burlingame, Calif.) and FITC-anti-CD64 (Becton Dickinson). After labeling, cells were analyzed with the LSRII system (BD Biosciences). Parallel studies contained isotype controls, all from Becton Dickinson. Positive control BD Compbeads (BD Bioscience) were labeled with the all fluorochrome-tagged antibodies. The labeled cells were analyzed with the FACSCalibur system (BD Biosciences, San Jose, Calif.).

The media from PBMCs cultured alone or stimulated with DM and/or MSCs after 5 days were quantitated for IFNγ and IL-10 at Cytokine Core Lab, University of Maryland, Baltimore, Md.

### Statistical Analyses

Data were analyzed using analysis of variance and Tukey-Kramer multiple comparisons test. We considered a p value less than or equal to 0.05 as significant.

### Results

### Antigenic Stimulation of Dust Mite on Responsive and Non-Responsive PBMCs

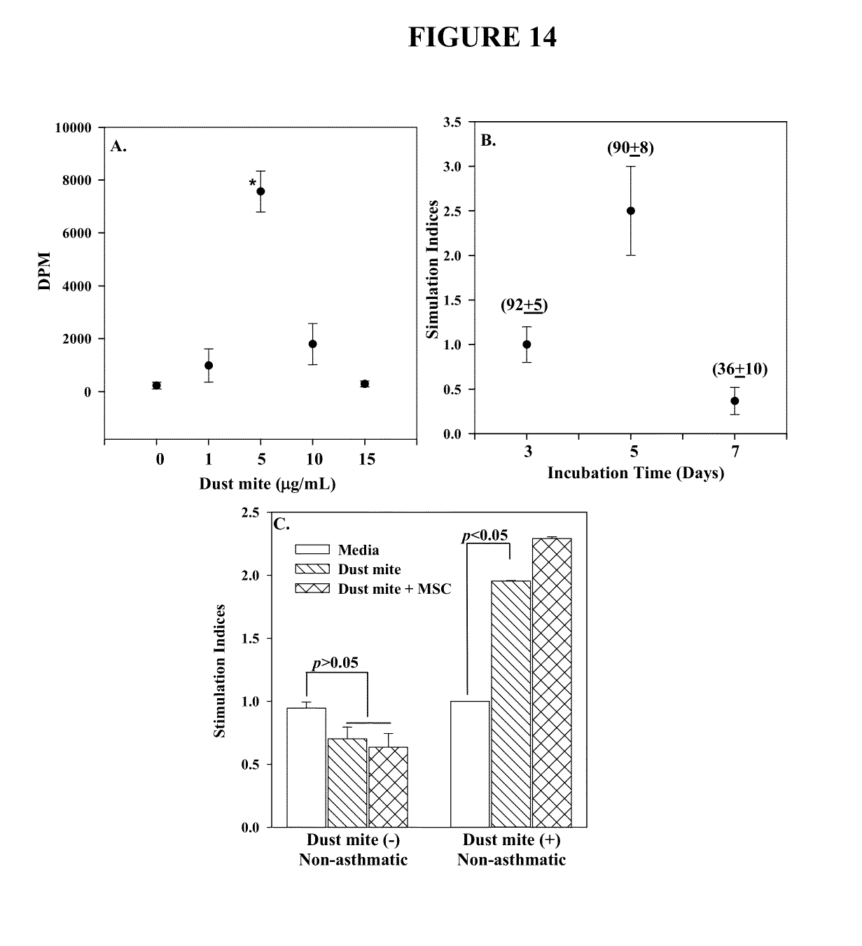

We first studied the specificity of dust mite antigen in proliferation of PBMCs. We compared four healthy controls with negative skin tests for dust mite to seven allergic asthmatic subjects, each studied in three independent experiments. The subjects who showed positive skin test and declared no history of asthma were designated ‘dust mite sensitive/non-asthmatic controls’ and were also included in the studies. The stimulation indices (S.I.) of dust mite-stimulated PBMCs from the subject with negative skin test were comparable (p>0.05) to media control (FIG. 14C, left group), indicating insensitivity/lack of proliferation of the PBMCs to dust mite challenge. In contrast, the subject with positive skin test showed significant (p<0.05) proliferation as compared to media control (FIG. 14, right group). The addition of MSCs did not show significant (p>0.05) effect on the proliferation of PBMCs (FIG. 14, right group, diagonal vs. hatched bars). In summary, this section shows the specificity of dust mite in the proliferation of PBMCs.

Effects of MSCs on the Proliferation of DM-Challenged PBMCs from Allergic Asthma

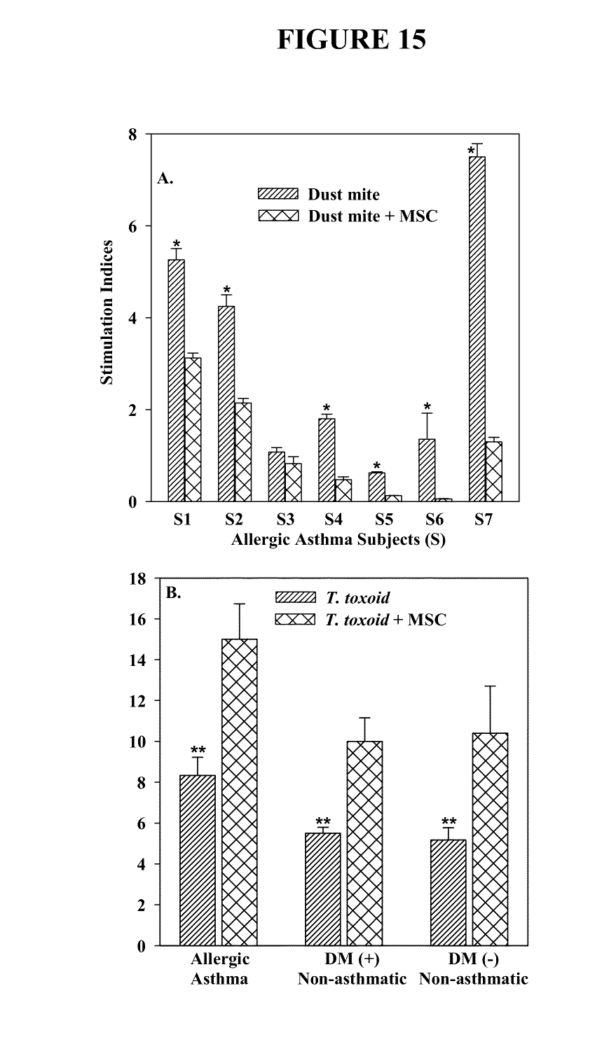

The studies described in FIG. 14 were repeated with the dust mite allergic asthma group with each subject studied in three independent experiments. In all subjects DM induced the proliferation of PBMCs (FIG. 15A, diagonal bars). MSCs significantly (p<0.05) reduced the proliferation for all subjects except subject 3 (S3) (FIG. 15A, hatched bar). The decreases for S1 and S2 were 1.6 and 1.7 fold, respectively. These suppressions were significantly (p<0.05) less than the other subjects with S6 showing a 19-fold decrease. In summary, MSCs significantly (p<0.01) decreased the proliferation PBMCs to DM from 6/7 allergic asthma subjects. Our subjects all received boost of T. toxoid. To determine if the effect on MSCs is antigen specific rather than allergen specific, we repeated the above studies with T. toxoid. In all cases proliferation was enhanced but further with MSCs (FIG. 15B). In summary, this section showed specificity of DM in the suppression of MSCs but only for PBMCs from allergic asthma.

T_{Regs} and DC in DM-Challenged PBMCs from Allergic Asthma Subjects

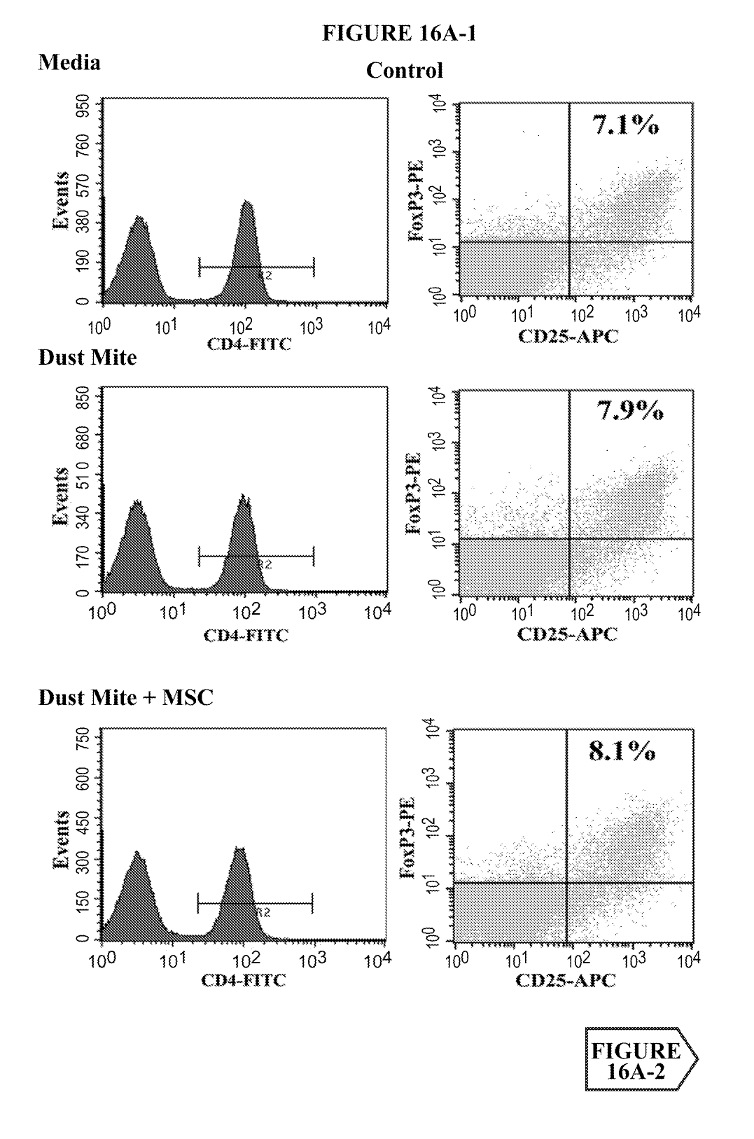

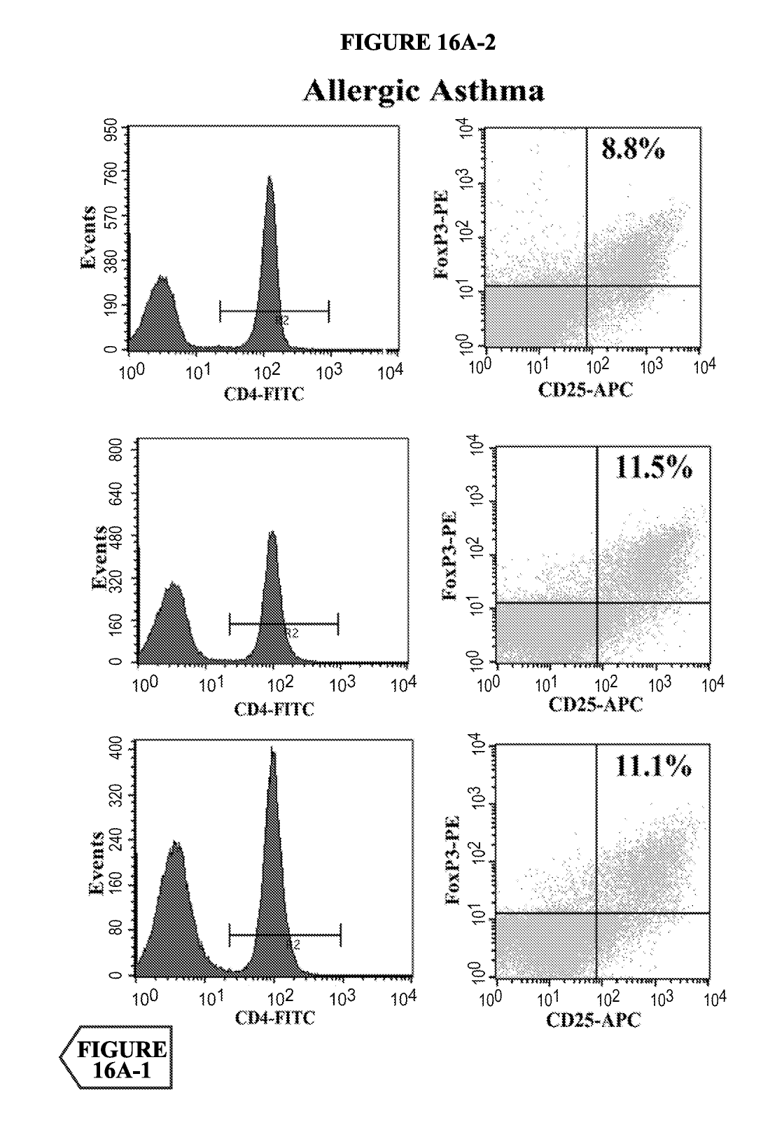

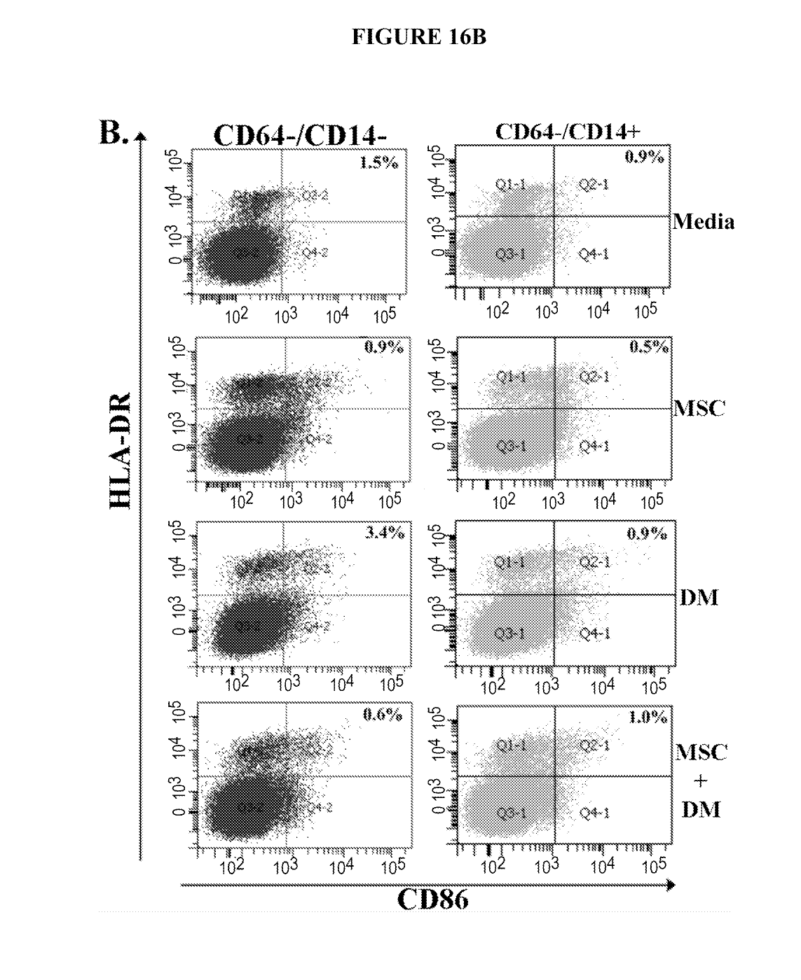

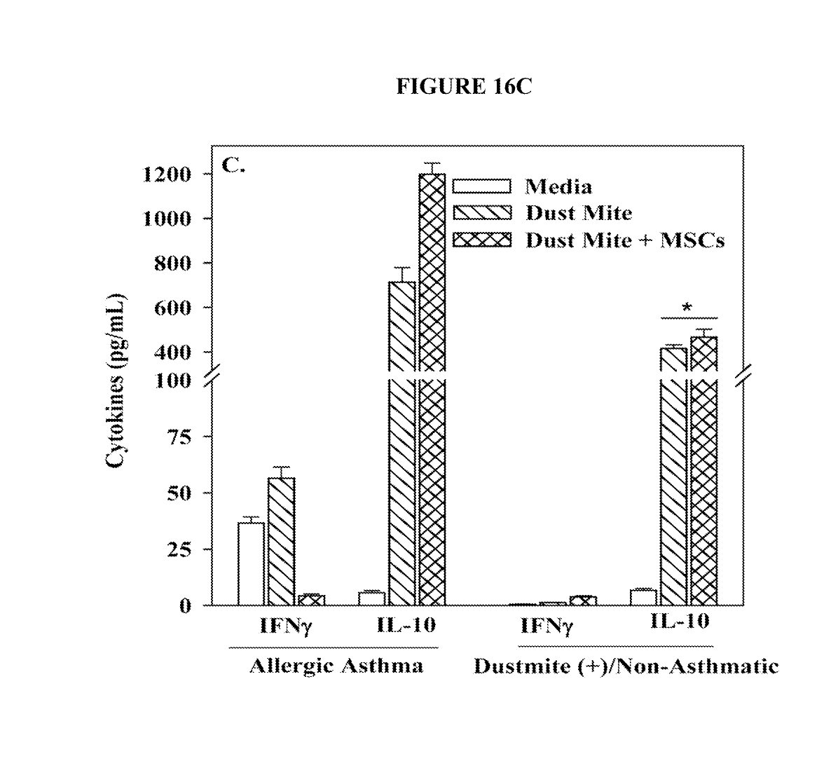

MSCs have been reported to cause immune suppression through secondary induction of T_{regs}^{22,24}. We therefore studied PBMCs, challenged for 5 days, in the presence or absence of MSCs, for T_{regs}. Flow cytometry for CD4, Foxp3 and CD25 revealed no significant (p>0.05) change in T_{reg} (FIG. 16A), although there appeared a trend towards an increase in T_{reg} in the allergic asthma subjects as compared to DM(−) non-asthmatic controls (left panels). Since Foxp3 and CD25 do not always prove the presence or absence of T_{regs}, IFNγ and IL-10 in the media were quantitated. The results showed significant increases in IL-10 but not IFNγ for both allergic asthma and allergic, non-asthmatic subjects (FIG. 16C). The IL-10 levels in the non-asthmatic group were significantly (p<0.05) less than similar conditions from the asthmatics group. Also, IFNγ remained low in the non-asthmatic group, despite stimulation with DM. In summary, the results showed no significant induction of T_{reg}, based on phenotype in the challenged AA subjects, regardless of MSCs, but showed an increase in IL-10 levels, indicating another mechanism of suppression.

Since it is unclear if T_{regs} are involved in the suppressive effects of MSCs (FIG. 16A), we studied the cultures for mature DCs. MSCs have been shown to inhibit monocyte-derived differentiation of DCs²⁵. Using the same experimental condition as for T_{reg} analyses, we performed multi-color flow cytometry for mature DC. We gated the CD64(−) fraction to eliminate granulocytes, and then performed further analyses, based on the expressions of CD14, HLA-DR and the mature co-stimulatory molecule CD86. In DM-treated PBMCs, there was about 6-fold increase in CD14−/CD86+ cells whereas no change was observed for the CD14+ fraction (FIG. 16B), indicating that MSCs blunted DM-induced maturation of DCs.

### Non-Responsiveness to DM by MSC-Preconditioned PBMC

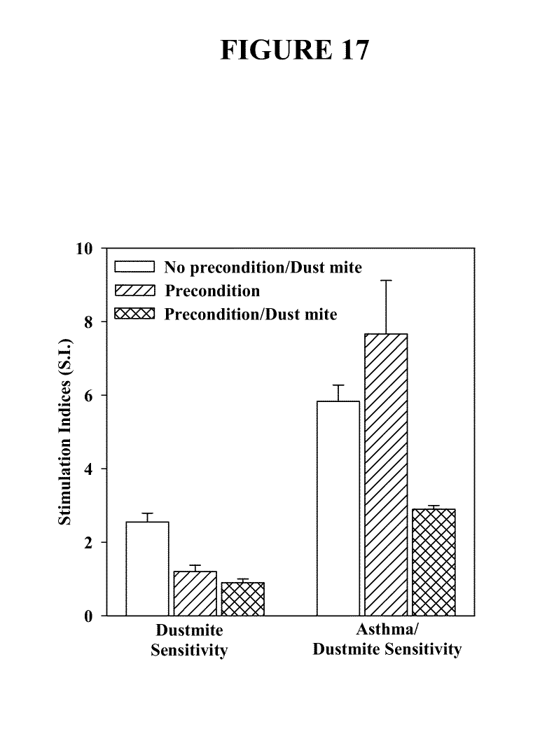

This set of studies describes the response of PBMCs, preconditioned with MSCs to DM. PBMCs were pre-incubated with MSCs and at day 5, the MSC-free PBMCs were studied in proliferation assays with DM. As expected, the unconditioned PBMCs responded to DM (FIG. 17, open bars). Interestingly, the preconditioned PBMCs from allergic asthma subjects proliferated when studied alone (FIG. 17, middle bar of right group), but this was significantly (p<005) reduced when rechallenged with DM (FIG. 17, left group, hatched bar). Similar treatment with the DM sensitive/non-asthmatic subjects did not show similar refractory to rechallenged DM (FIG. 17, left group, hatched bar).

We next asked if preconditioning requires direct contact between PBMCs and MSCs. This was addressed in transwell cultures with MSCs in the inner wells and PBMCs in the outer wells, in the presence or absence of optimal DM. After 5 days, the PBMCs were studied in proliferation assay, as above. The PBMCs responded to DM with S.I., 6±0.8, indicating that preconditioning of PBMCs from allergic asthma requires direct contact with MSC.

Refractoriness of PBMCs by Repeated Challenge with Dust Mite Antigen and MSCs

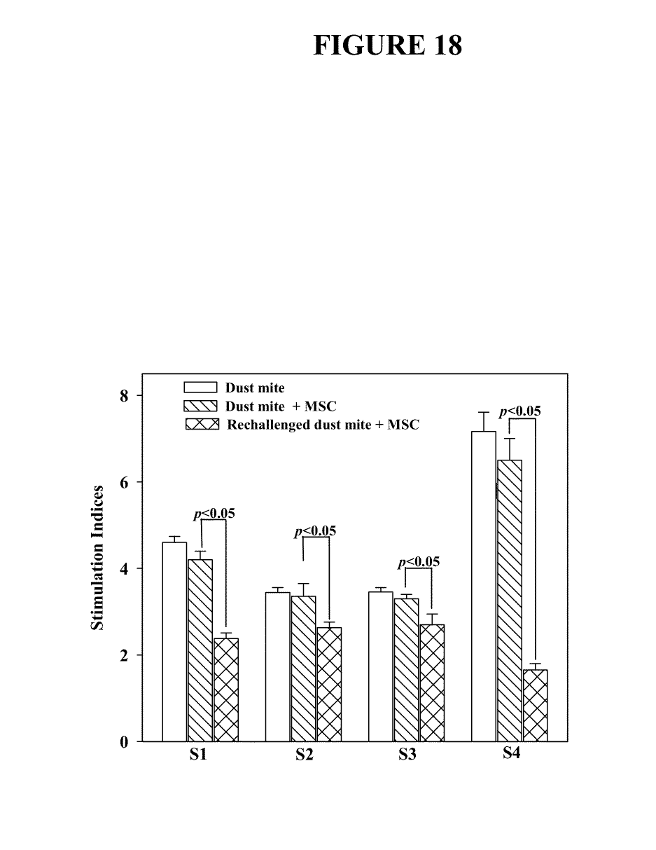

Refractoriness of the preconditioned PBMCs, together with a blunting effect on DC maturation (FIGS. 16B, 17) led us to ask if we can induce refractoriness with repeated low doses of DM, in the presence of MSCs. In four different subjects, each studied in three independent experiments, were rechallenged with optimal DM. Indeed, the S.I. of PBMCs were significantly decreased indicating refractoriness of the repeated challenge cells, similar to what would be expected of anergy (FIG. 18). The suppression could not be due to the FCS in the cultures, since parallel cultures with DM alone were able to respond to DM (FIG. 18, open bars).

### Discussion

The literature has reported on dual immune functions of MSCs, immune suppression and enhancement^{23,26}. Each type of response appears to occur during a narrow window, depending on the milieu^{21,23}. In this study, we observed a veto-like response since MSCs showed preference by suppressing PBMCs from allergic asthma subjects that are expected to be hyperactivated as compared to subjects with only allergic responses, mono- and poly-sensitized (FIGS. 14C, 15B). The results are not antigen-specific, but for allergens, as denoted by studies with T. toxoid (FIG. 15B). In the presence of MSCs, T. toxoid showed enhanced proliferation (FIG. 15B). We expect that this can be explained by the antigen presenting properties of MSCs²³.

PBMCs from subjects with allergic asthma showed significantly more response to DM as compared to similar challenges of PBMCs from DM(+) non-asthmatic (FIGS. 14C and 15). This type of outcome mimics a veto-like response since it is expected that PBMCs from allergic asthma would be more sensitive to stimulation with an allergen²³. In contrast to the inhibitory effect of MSCs on the proliferation of PBMCs from allergic asthma subjects, MSCs could not suppress DM-activated PBMCs from non-asthmatic/DM sensitive subjects (FIG. 14C). These findings are consistent with other studies in which MSCs could not suppress T. toxoid-challenged PBMCs²¹. In fact, the addition of MSCs to the non-asthmatic/dust mite (+) PBMCs showed a trend towards increased proliferation of the PBMCs (FIG. 14/right group/hatched bar). We explain this finding by other studies showing an antigen presenting role of MSCs when IFN-γ level is low²³. It is possible that the relatively low proliferative response to dust mite prevented high level of IFNγ for antigen presenting function.

The suppressive effect of MSCs on the proliferation of PBMCs from allergic asthma subjects recapitulates inflammation, which would cause decreases in cytokine production. These findings are relevant to the translation of MSCs for allergic asthma, in light of the deleterious secondary effects of cytokines in allergic asthma²⁷.

MSC-mediated immune suppression has been linked to increases in T_{regs} and related cytokines^{2,28,29}. In this study, the level of T_{regs} was unchanged with MSCs and DM (FIG. 16A). We correlated these findings with specific cytokines and found an increase in IL-10 with no change in IFNγ for the DM/non-asthmatic group (FIG. 16C). In other studies, T_{reg} induction has been shown to reduce airway inflammation, caused by DM³⁰. Since these studies were done in vivo the authors can examine the mice for long periods. It is possible that our 5-day model might be inadequate to induce T_{regs}. It is difficult to retain the cultures for longer periods without a high rate of cell death. The studies by Leech et al³⁰ were done only with DM, which caused reduced responses in non-asthmatic subjects, indicating differences in the two models. We believe that the inability of MSCs to induce T_{regs}, despite its suppressive effect, is an advantage for translation in allergic asthma. If MSCs induce T_{regs} this could cause untoward effects such as those linked to immune suppressor drugs. T_{regs} have been reported to suppress T-cell responses by absorbing the proinflammatory IL-2³¹. Since this method could not occur in our model, the effect of MSCs is by other mechanisms. In our studies, we have observed a blunt effect of MSCs in the maturation of DCs (FIG. 16B).

The mechanism and consequence of reduced mature DC in the presence of DM and MSCs require further exploration (FIG. 16B). While the literature overwhelmingly showed evidence of MSC-mediated suppression through impediment of DC maturation^{29,32-34}, this model is different since the blunting effect is within a milieu of an allergen. MSCs can down regulate the expression of activating receptors on natural killer cells³⁵.

This study also provides a method by which tolerance to allergen is achieved. It is expected that MSCs will provide long-lasting tolerance to allergens, beyond DM. We have shown refractoriness of PBMCs to rechallenged DM by two methods, preconditioning with MSCs and repeated exposure to low dose DM and MSCs (FIGS. 17 and 18).

The varied responses of dust mite-challenged PBMCs to MSCs could not be explained by the subjects' body mass indices (BMI), although the answer to this question would benefit from a larger cohort. This question is pertinent since adipose tissues are a rich source of MSCs¹⁶. If PBMCs from obese patients are exposed to adipose-derived MSCs, this might influence the allergic response and perhaps asthmatic effects. In this study, grouping of the subjects by BMI, i.e., between 20-30 and 31-40, as a function of fold-decrease in PBMC proliferation showed no conclusive outcome.

In summary, this pilot study showed a complex role for MSCs in allergic disease. The findings are significant to allergic asthma. Although we used dust mite as a model system, the findings can be extrapolated to other allergens.

TABLE 2
Demographics of patient population in the dust mite allergic asthma group

| | | | FEV1/ | | | | Mono/ | | |
|---|---|---|---|---|---|---|---|---|---|
| | Age | | FVC | FEV1 | Atopic | Total | Poly- | | Controller |
| Subjects | (yrs) | Ethnicity | (%) | (%) | Diseases | IgE | sensitized | BMI | medications |
| 1 | 38 | AA | 111 | 100 | AR/AC | NA | mono | 38.6 | Fluticasone |
| | | | | | | | | | 220 μg |
| 2 | 21 | AA | 95 | 103 | AR/AC | NA | poly | 38.3 | Budesonide |
| | | | | | eczema | | | | |
| 3 | 40 | AA | 98 | 91 | AR/AC | NA | poly | 28.3 | Budesonide |
| 4 | 23 | H | 91 | 88 | AR/AC | 1187 | poly | 23.9 | Albuterol |
| 5 | 33 | H | 75 | 100 | AR chronic | NA | mono | 40.6 | Ciclesonide |
| | | | | | sinusitis | | | | |
| 6 | 39 | C | 83 | 86 | AR smoker | NA | poly | 34.3 | Fluticasone/ |
| | | | | | | | | | Salmeterol |
| | | | | | | | | | 500/50 μg |
| | | | | | | | | | Montelukast |
| | | | | | | | | | Levo- |
| | | | | | | | | | cetirizine |
| 7 | 23 | M | 98 | 96 | AR/AC | NA | poly | 25.1 | Albuterol (as |
| | | | | | | | | | needed) |

AR: allergic rhinitis;
AC: allergic conjunctivitis;
NA: not available;
AA: African American;
H: Hispanic;
C: Caucasian;
M: Mixed

The experiments disclosed in this Example establish that mesenchymal stem cells (MSCs), through intercellular contact with peripheral blood mononuclear cells (PBMCs) from allergic asthma, suppressed the response of PBMCs to dust mite challenge. The suppressive effects of MSCs correlated with an increase in mature dendritic cells, a decrease in IFNγ, and an increase in IL-10. PBMCs gained refractoriness by preconditioning with MSCs and by repeated exposure to low dose DM and MSCs, indicating that MSCs have the potential to reset the immune system for allergic asthma.

### References for Example 2

1. Rasmusson I. Immune modulation by mesenchymal stem cells. Exp Cell Res 2006; 312:2169-79.
2. Uccelli A, Moretta L, Pistoia V. Mesenchymal stem cells in health and disease. Nat Rev Immunol 2008; 8:726-36.
3. Prockop D J, Kota D J, Bazhanov N, Reger R L. Evolving paradigms for repair of tissues by adult stem/progenitor cells (MSCs). J Cell Mol Med 2010; 14:2190-9.
4. Maitra B, Szekely E, Gjini K, Laughlin M J, Dennis J, Haynesworth S E et al. Human mesenchymal stem cells support unrelated donor hematopoietic stem cells and suppress T-cell activation. Bone Marrow Transplant 2004; 33:597-604.
5. Tse W T, Pendleton J D, Beyer W M, Egalka M C, Guinan E C. Suppression of allogeneic T-cell proliferation by human marrow stromal cells: implications in transplantation. Transplantation 2003; 75:389-97.
6. Liang J, Gu F, Wang H, Hua B, Hou Y, Shi S et al. Mesenchymal stem cell transplantation for diffuse alveolar hemorrhage in SLE. Nat Rev Rheumatol 2010; 6(8):486-9.
7. Sun L, Akiyama K, Zhang H, Yamaza T, Hou Y, Zhao S et al. Mesenchymal Stem Cell Transplantation Reverses Multiorgan Dysfunction in Systemic Lupus Erythematosus Mice and Humans. Stem Cells 2009; 27:1421-32.
8. Gonzalez-Rey E, Gonzalez M A, Varela N, O'Valle F, Hernandez-Cortes P, Rico L et al. Human adipose-derived mesenchymal stem cells reduce inflammatory and T cell responses and induce regulatory T cells in vitro in rheumatoid arthritis. Ann Rheumatic Dis 2010; 69:241-8.
9. Zheng Z H, Li X Y, Ding J, Jia J F, Zhu P. Allogeneic mesenchymal stem cell and mesenchymal stem cell-differentiated chondrocyte suppress the responses of type II collagen-reactive T cells in rheumatoid arthritis. Rheumatol 2008; 47:22-30.
10. Zhang Q, Shi S, Liu Y, Uyanne J, Shi Y, Shi S et al. Mesenchymal stem cells derived from human gingiva are capable of immunomodulatory functions and ameliorate inflammation-related tissue destruction in experimental colitis. J Immunol 2010; 184:1656.
11. Garcia-Olmo D, Garcia-Arranz M, Herreros D, Pascual I, Peiro C, Rodriguez-Montes J A. A phase I clinical trial of the treatment of Crohn's fistula by adipose mesenchymal stem cell transplantation. Dis Colon Rectum 2005; 48:1416-23.
12. Parekkadan B, Tilles A W, Yarmush M L. Bone Marrow-Derived Mesenchymal Stem Cells Ameliorate Autoimmune Enteropathy Independently of Regulatory T Cells. Stem Cells 2008; 26:1913-9.
13. Bartholomew A, Sturgeon C, Siatskas M, Ferrer K, McIntosh K, Patil S et al. Mesenchymal stem cells suppress lymphocyte proliferation in vitro and prolong skin graft survival in vivo. Exp Hematol 2002; 30:42-8.
14. Ringden O, Uzunel M, Rasmusson I, Remberger M, Sundberg B, Lonnies H et al. Mesenchymal stem cells for treatment of therapy-resistant graft-versus-host disease. Transplantation 2006; 81:1390-7.
15. Tyndall A, Pistoia V. Mesenchymal stem cells combat sepsis. Nat Med 2009; 15:18-20.
16. Salem H K, Thiemermann C. Mesenchymal Stromal Cells: Current Understanding and

Clinical Status. Stem Cells 2010; 28:585-96.
17. Helmy K Y, Patel S A, Silverio K, Pliner L, Rameshwar P. Stem cells and regenerative medicine: accomplishments to date and future promise. Ther Deliv 2010; 1:693-705.
18. Lun S, Wong C, Ko F, Hui D, Lam C. Expression and Functional Analysis of Toll-Like Receptors of Peripheral Blood Cells in Asthmatic Patients: Implication for Immunopathological Mechanism in Asthma. J Clin Immunol 2009; 29:330-42.
19. Park H K, Cho K S, Park H Y, Shin D H, Kim Y K, Jung J S et al. Adipose-Derived Stromal Cells Inhibit Allergic Airway Inflammation in Mice. Stem Cells Dev 2010; 19:1811-8.
20. Kim H Y, DeKruyff R H, Umetsu D T. The many paths to asthma: phenotype shaped by innate and adaptive immunity. Nat Immunol 2010; 11:577-84.
21. Potian J A, Aviv H, Ponzio N M, Harrison J S, Rameshwar P. Veto-Like Activity of Mesenchymal Stem Cells: Functional Discrimination Between Cellular Responses to Alloantigens and Recall Antigens. J Immunol 2003; 171:3426-34.
22. Patel S A, Meyer J R, Greco S J, Corcoran K E, Bryan M, Rameshwar P. Mesenchymal Stem Cells Protect Breast Cancer Cells through Regulatory T Cells: Role of Mesenchymal Stem Cell-Derived TGF-beta. J Immunol 2010; 184:5885-94.
23. Chan J L, Tang K C, Patel A P, Bonilla L M, Pierobon N, Ponzio N M et al. Antigen-presenting property of mesenchymal stem cells occurs during a narrow window at low levels of interferon-gamma. Blood 2006; 107:4817-24.
24. Di Ianni M, Del Papa B, De Ioanni M, Moretti L, Bonifacio E, Cecchini D et al. Mesenchymal cells recruit and regulate T regulatory cells. Exp Hematol 2008; 36:309-18.
25. Spaggiari G M, Abdelrazik H, Becchetti F, Moretta L. MSCs inhibit monocyte-derived DC maturation and function by selectively interfering with the generation of immature DCs: central role of MSC-derived prostaglandin E2. Blood 2009; 113:6576-83.
26. Francois M r, Romieu-Mourez R1, Stock-Martineau S, Boivin M N1, Bramson J L, Galipeau J. Mesenchymal stromal cells cross-present soluble exogenous antigens as part of their antigen-presenting cell properties. Blood 2009; 114:2632-8.
27. Bullens D M. Measuring T cell cytokines in allergic upper and lower airway inflammation: can we move to the clinic? Inflamm Allergy Drug Targets 2007; 6:81-90.
28. Nouri-Aria K T, Durham S R. Regulatory T cells and allergic disease. Inflamm Allergy Drug Targets 2008; 7:237-52.
29. Ramasamy R, Fazekasova H, Lam E W, Soeiro I, Lombardi G, Dazzi F. Mesenchymal stem cells inhibit dendritic cell differentiation and function by preventing entry into the cell cycle. Transplantation 2007; 83:71-6.
30. Leech M D, Benson R A, deVries A, Fitch P M, Howie S E M. Resolution of Der p1-Induced Allergic Airway Inflammation Is Dependent on CD4+CD25+Foxp3+ Regulatory Cells. J Immunol 2007; 179:7050-8.
31. Busse D, de la Rosa M, Hobiger K, Thurley K, Flossdorf M, Scheffold A et al. Competing feedback loops shape IL-2 signaling between helper and regulatory T lymphocytes in cellular microenvironments. Proc Natl Acad Sci 2010; 107:3058-63.
32. Jiang X X, Zhang Y, Liu B, Zhang S X, Wu Y, Yu X D et al. Human mesenchymal stem cells inhibit differentiation and function of monocyte-derived dendritic cells. Blood 2005; 105:4120-6.
33. Nauta A J, Kruisselbrink A B, Lurvink E, Willemze R, Fibbe W E. Mesenchymal Stem Cells Inhibit Generation and Function of Both CD34+-Derived and Monocyte-Derived Dendritic Cells. J Immunol 2006; 177:2080-7.
34. Li Y P, Paczesny S, Lauret E, Poirault S, Bordigoni P, Mekhloufi F et al. Human Mesenchymal Stem Cells License Adult CD34+ Hemopoietic Progenitor Cells to Differentiate into Regulatory Dendritic Cells through Activation of the Notch Pathway. J Immunol 2008; 180:1598-608.
35. Spaggiari G M, Capobianco A, Becchetti S, Mingari M C, Moretta L. Mesenchymal stem cell-natural killer cell interactions: evidence that activated NK cells are capable of killing MSCs, whereas MSCs can inhibit IL-2-induced NK-cell proliferation. Blood 2006; 107:1484-90.
36. Glennie S, Soeiro Is, Dyson P J, Lam E W F, Dazzi F. Bone marrow mesenchymal stem cells induce division arrest anergy of activated T cells. Blood 2005; 105:2821-7.
37. Benvenuto F, Ferrari S, Gerdoni E, Gualandi F, Frassoni F, Pistoia V et al. Human Mesenchymal Stem Cells Promote Survival of T Cells in a Quiescent State. Stem Cells 2007; 25:1753-60.
38. Ren G, Zhang L, Zhao X, Xu G, Zhang Y, Roberts A I et al. Mesenchymal Stem Cell-Mediated Immunosuppression Occurs via Concerted Action of Chemokines and Nitric Oxide. Cell Stem Cell 2008; 2:141-50.
39. Munn D H, Shafizadeh E, Attwood J T, Bondarev I, Pashine A, Mellor A L. Inhibition ofΓ [symbol] ë T Cell Proliferation by Macrophage Tryptophan Catabolism. J Exp Med 1999; 189:1363-72.
40. Hwu P, Du M X, Lapointe R+, Do M, Taylor M W, Young H A. Indoleamine 2,3-Dioxygenase Production by Human Dendritic Cells Results in the Inhibition of T Cell Proliferation. J Immunol 2000; 164:3596-9.
41. Frumento G, Rotondo R, Tonetti M, Damonte G, Benatti U, Ferrara G B. Tryptophan-derived Catabolites Are Responsible for Inhibition of T and Natural Killer Cell Proliferation Induced by Indoleamine 2,3-Dioxygenase. J Exp Med 2002; 196:459-68.
42. Krampera M, Cosmi L, Angeli R, Pasini A, Liotta F, Andreini A et al. Role for Interferon-gamma in the Immunomodulatory Activity of Human Bone Marrow Mesenchymal Stem Cells. Stem Cells 2006; 24:386-98.
43. Ryan J M, Barry F, Murphy J M, Mahon B P. Interferon-gamma does not break, but promotes the immunosuppressive capacity of adult human mesenchymal stem cells. Clin Exp Immunol 2007; 149:353-63.
44. Luckey U, Maurer M, Schmidt T, Lorenz N, Seebach B, Metz M et al. T cell killing by tolerogenic dendritic cells protects mice from allergy. J Clin Invest 2011; 121:3860-71.

### Example 3

Mesenchymal stem cells (MSCs) are promising cellular suppressors of inflammation. This function of MSCs is partly due to their licensing by inflammatory mediators. In cases with reduced inflammation, MSCs could become immune-enhancer cells. MSCs can suppress the inflammatory response of antigen-challenged lymphocytes from allergic asthma. Although allergic rhinitis (AR) is also an inflammatory response, it had been unclear if MSCs could exert similar suppression. This study investigated the immune effects (suppressor vs. enhancer) of MSCs on allergen-stimulated lymphocytes from AR subjects (grass or weed allergy). In contrast to subjects with allergic asthma, MSCs caused a significant (p<0.05) increase in the proliferation of antigen-challenged lymphocytes from AR subjects. The increase in lymphocyte proliferation was caused by the MSCs presenting the allergens to CD4+ T-cells (APCs). This correlated with increased production of inflammatory cytokines from T-cells, and increased expressions of MHC-II and CD86 on MSCs. The specificity of APC function was demonstrated in APC assay using MSCs that were knocked down for the master regulator of MHC-II transcription, CIITA. The difference in the effects of MSCs on allergic asthma and AR could not be explained by the sensitivity to the allergen, based on skin tests. Thus, we deduced that the contrasting immune effects of MSCs for antigen-challenged lymphocytes on AR and allergic asthma could be disease-specific. It is possible that the enhanced inflammation from asthma might be required to license the MSCs to become suppressor cells.

Mesenchymal stem cells (MSCs) differentiate along various lineages to generate specialized cells of all germ layers, such as cartilage, muscle, neurons and cardiomyocytes^{1,2}. MSCs are attractive for cell therapy, due to ease in expansion, reduced ethical concerns and low probability of transformation^{2,3}. MSCs are ubiquitous and are referred by different names such as pericytes⁴. Regardless of the source, MSCs generally show similarity with respect to immune functions. Phenotypic and functional studies indicate that MSCs are heterogeneous⁵. Without wishing to be bound by theory, one possible explanation for MSC heterogeneity is the co-existence of stem cells and progenitors within the MSC culture. Until reagents are developed that establish a hierarchy of MSCs, the methods used in a particular study will require detailed documentation for the appropriate interpretation of the results.

MSCs are expected to be effective anti-inflammatory cells for treating multiple sclerosis, asthma, graft-versus-host disease, Crohn's disease and other inflammatory disorders¹⁷⁻²⁰. The microenvironmental milieu is a factor in determining whether MSCs act as immune suppressor or enhancer cells^{21,22}. The mechanism by which MSCs exert immune suppression is complex, occurring partly through soluble factors such as cytokines, indoleamine 2,3 dioxygenase, hepatocyte growth factor, prostaglandin E2, and nitric oxide²³⁻²⁶. MSCs can alter the functions of T-cells, dendritic cells and natural killer cells by switching the production of cytokines to alter T-cell response^{17,23}. MSCs also respond to chemotactic factors and migrate to areas of inflammation²⁷.

Among the immune enhancing roles of MSCs is their ability to be antigen presenting cells (APCs). This function is tightly controlled by the level of IFNγ^{28,29}. This occurs by the controlled expression of MHC-II³⁰. IFNγ controls the APC function of MSCs by regulating the expression of the master regulator of MHC-II, CIITA³¹. At high levels of IFNγ, CIITA is retained in the cytosol to prevent the transcription of MHC-II whereas low levels of IFNγ caused nuclear retention of CIITA³¹. MSCs can also cross-present antigen by cross-presentation as another aspect of their APC function³².

MSCs suppressed inflammatory responses in animal models of allergic airway inflammation and ragweed-induced asthma^{33,34}. Despite the surge of research on MSCs as anti-inflammatory cells, there is little information on MSCs as immune suppressors. The experiments disclosed herein investigated the immune effects of MSCs for allergic rhinitis (AR) because AR remains one of the most prevalent inflammatory conditions.

AR is a common chronic inflammatory disease of the nasal passages, affecting approximately 20% of adults in the United States³⁵. AR is an allergic disease characterized by an influx of eosinophils and Th2 cells, increases in pro-inflammatory cytokines such as IL-3, IL-4, IL-5, IL-13, influx of eosinophils and IgE-mediated mast cell degranulation³⁶. Immune hyper-reactivity to common environmental allergens typically cause chronic nasal congestion, rhinorrhea, sneezing, postnasal drip and, itchy/watery. AR worsens other conditions, such as asthma and sinusitis, and increases health care costs³⁷. In an animal model of AR, transplanted MSCs migrated to the nasal mucosa to reduce the symptoms to decrease infiltrating eosinophil and reduced sera IgE^{17,38}.

Consideration of the dual immune roles of MSCs leads to the realization that MSCs could also worsen the inflammatory state of AR^{28-30,32}. To this end, this study investigated a role for MSCs in antigen-challenged peripheral blood mononuclear cells (PBMCs) from patients with AR. The results establish an enhanced immune effect of MSCs to pollen provocation with PBMCs from AR. The studies showed enhancement in the immune response, due to the APC role of MSCs.

### Results

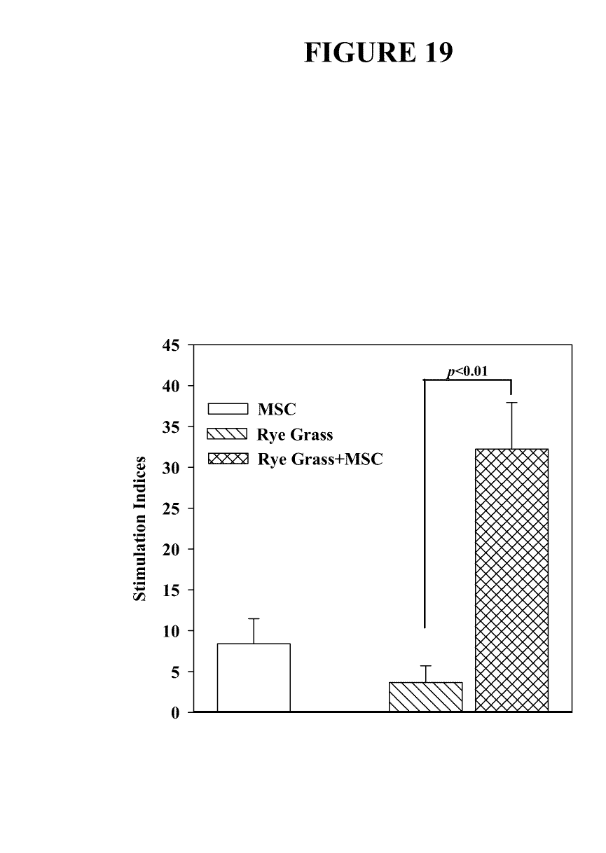

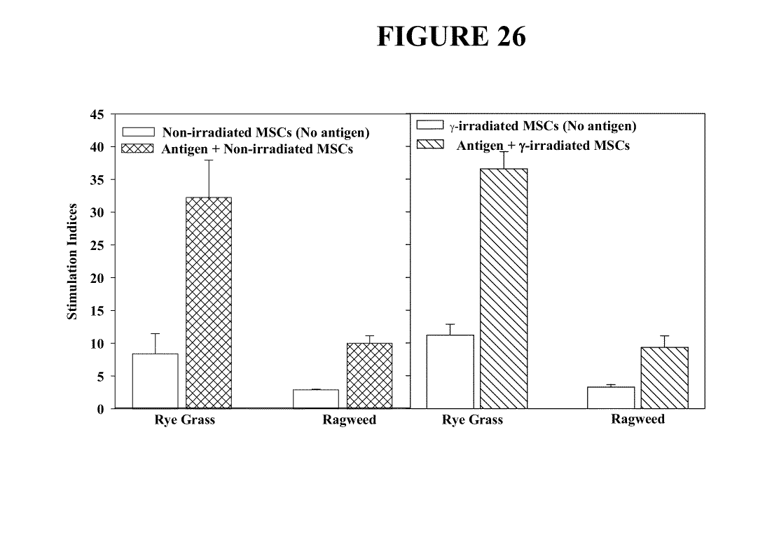

The low expression of MHC-II on MSCs are sufficient to elicit allogeneic responses²². To account for the contribution of allogeneic differences between the MSCs and the test PBMCs, all of the studies included controls consisting of one-way mixed reactions with MSCs as stimulators and PBMCs as responders (FIGS. 19-21, open bars). Studies with γ-irradiated MSCs (2000 Rads) vs. non-irradiated MSCs confirmed that the proliferation observed in the mixed cultures was indeed due solely to the PBMCs and not the MSCs (FIG. 26).

MSCs Increased the Proliferation of PBMCs Challenged with Rye Grass

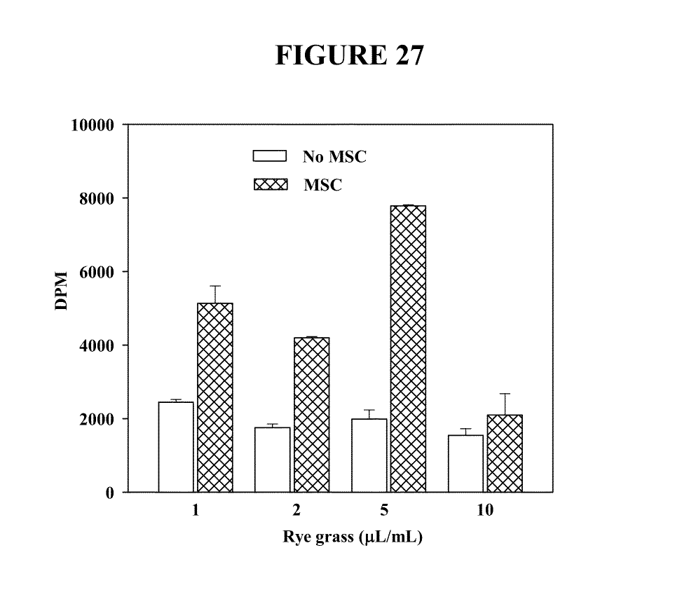

We studied the effects of MSCs on the proliferation of antigen-challenged PBMCs from subjects with rye grass sensitivity. First we tested for the optimum concentration of rye grass in dose response studies with antigen alone or with antigen and MSCs. Studies with six donors and rye grass between 1 and 20 μl/ml indicated 5 μl/ml as the optimum concentration (FIG. 27). This information was based on the effect of PBMCs and MSCs rather than PBMCs alone.

The allogeneic differences between PBMCs with MSCs were verified in mixed cultures with MSCs as the stimulators and PBMCs as the responders^{22,28,30,39}. The results indicated stimulation indices (S.I.) of 8.4±3 (±SD), which signified allogeneic differences (FIG. 19, open bar). PBMCs stimulated with optimal rye grass resulted in S.I.=3.7±2 (±SD) (FIG. 19, diagonal bar). Since the S.I. was calculated as the proliferation of antigen stimulated PBMCs/unstimulated PBMCs, the increase in S.I. verified sensitivity to rye grass. Despite the increase, the sensitivity to rye grass was nonetheless relatively low. The addition of MSCs and rye grass antigen showed a significant (p<0.01) increase in the S.I. to 32±5.7 (±SD) (FIG. 19, hatched bar). This increase was about 8-fold more than the cultures with antigen alone (FIG. 19, diagonal bar). In summary, the data showed an enhancing effect of MSCs in rye-challenged PBMCs from AR subjects.

Effects of MSCs on Ragweed-Challenged PBMCs from AR Subjects

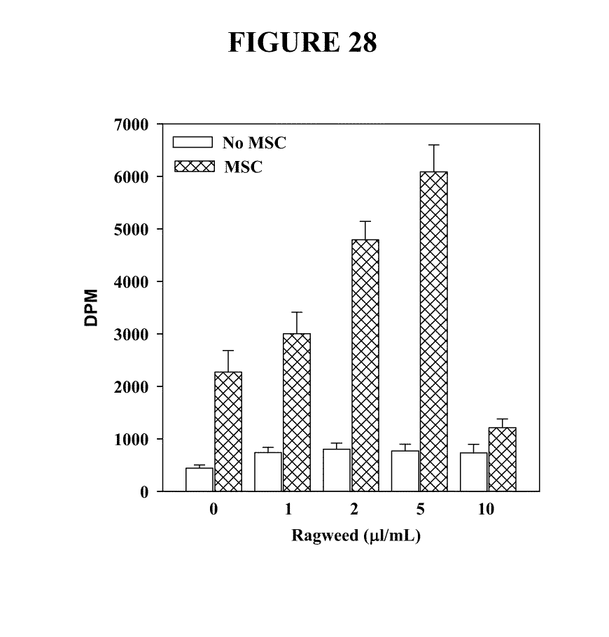

The immune-enhancing effect of MSCs in the rye grass-challenged cultures was unexpected because MSCs can suppress inflammatory responses^{22,40,41}. We therefore examined another allergen to determine if the enhancing effect was limited to rye grass. We selected ragweed sensitivity from six donors. Dose response studies with different concentrations of ragweed indicated the optimum concentration to be 5 μL/mL (FIG. 28).

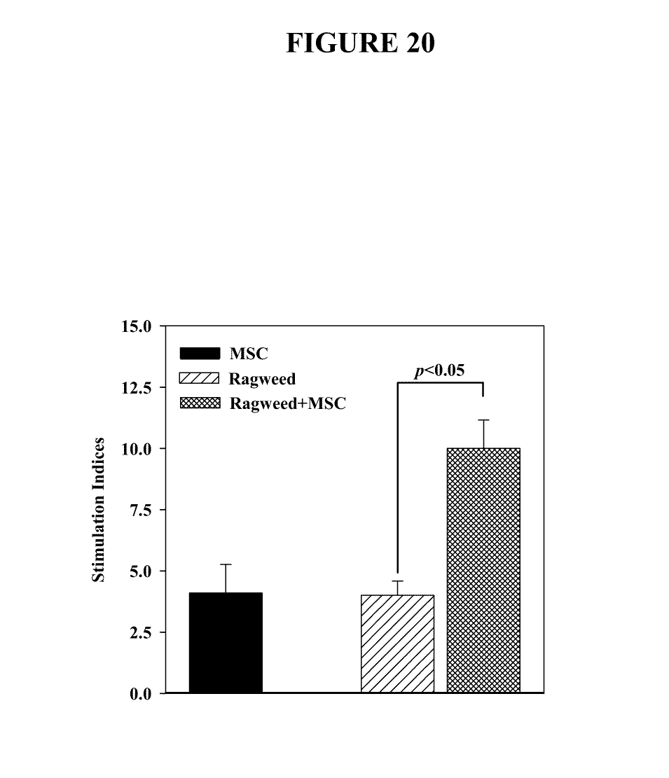

PBMCs from the same donors used for the dose-response studies were stimulated with optimum ragweed, in the absence or presence of MSCs. Baseline proliferation was assessed with PBMCs alone. PBMCs and MSCs (allogeneic response) and PBMCs and ragweed showed comparable proliferation (p>0.05) (FIG. 20, solid and diagonal bars). However, ragweed and MSCs, resulted in significant (p<0.05) increase in the S.I. as compared to the other two experimental points (FIG. 20, hatched bar). In summary, we showed an enhancing effect of MSCs to ragweed-challenged PBMCs. This contrasted the modest in vitro responses to pollen alone.

Effects of MSCs in Ragweed-Challenged PBMCs from Allergic Asthma

We previously reported on a suppressive effect of MSCs on allergic asthma using dust mite antigen²⁰. This was unlike what we observed for the non-asthmatic AR shown in FIGS. 19 and 20. We therefore selected subjects with asthma who were allergic to ragweed and rye grass to determine if the immune-enhancing effect of MSCs was caused by the allergen and not the underlying inflammatory condition (AR versus allergic asthma). Furthermore, since we previously studied allergic asthma with dust mite, the section will answer if the effect of MSCs was antigen-specific²⁰.

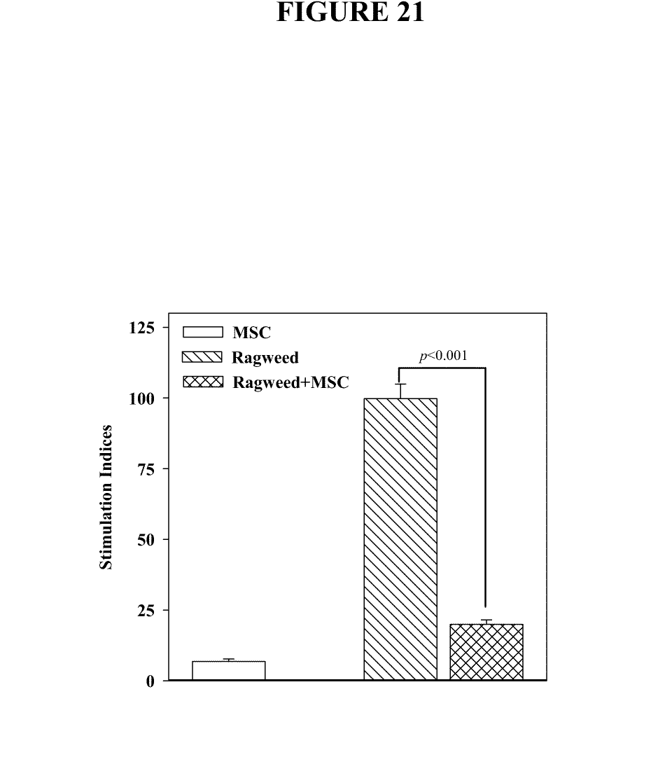

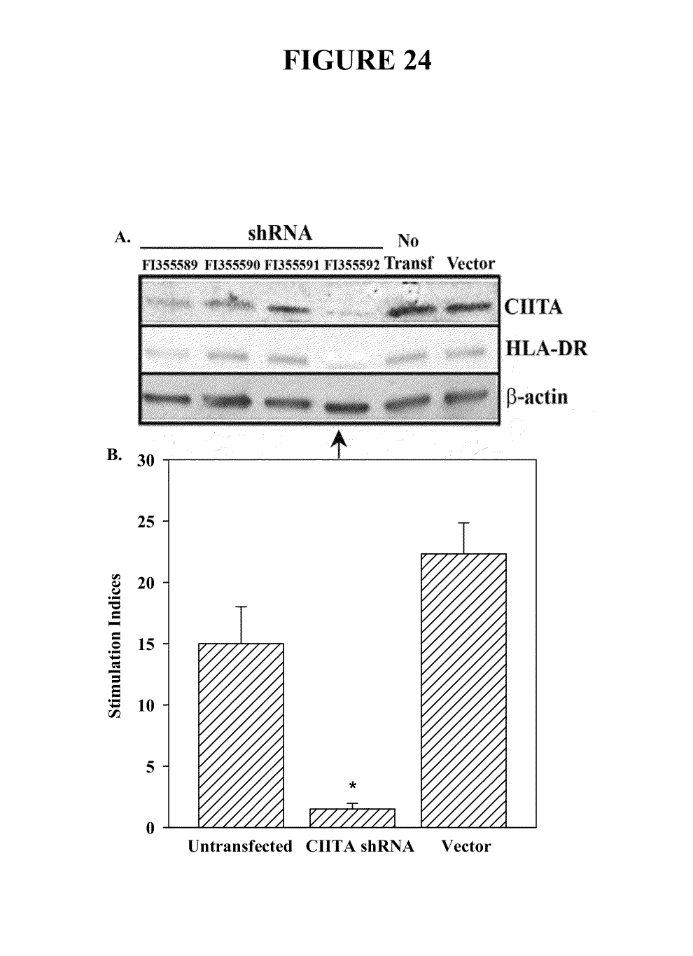

Proliferation studies were established as for FIGS. 19 and 20 with PBMCs from subjects with allergic asthma and sensitivity to ragweed, in the presence or absence of MSCs. The proliferation (S.I.) of PBMCs stimulated with ragweed was 103±8 (FIG. 21, diagonal bar). The proliferation was reduced (p<0.001) by 4.8 folds in the presence of MSCs (FIG. 21, hatched bar). The suppressive role of MSCs for AR subjects contrasted the immune-enhancing effect noted for AR subjects for both rye grass and ragweed (FIGS. 19 and 20). These results, combined with our demonstration that CIITA knockdown decreased HLA-DR expression (FIG. 24A), confirmed the essential role of the APC functionality of MSCs in the observed effects on T-cell proliferation in the context of AR²⁰.

### Antigen Presentation (APC) of Antigen-Challenged MSCs

MSCs can exert immune enhancer functions such as APCs and cross presentation by MHC-I^{29,30,32}. We proposed similar immune enhancing effect occurred for the proliferation observed for antigen challenged PBMCs from AR subjects (FIGS. 19 and 20). The literature reported on immune-enhancing outcome when the inflammatory milieu cannot adequately license MSCs to become immune suppressor cells³⁰. We studied if MSCs can exert APC function when challenged with ragweed or rye grass.

PBMCs from AR subjects were primed with rye grass or ragweed for five days. The CD4+ T-cells were selected and then added to antigen-pulsed MSCs. Control cultures contained activated CD4+ T-cells alone or with unpulsed MSCs. Baseline proliferation contained unactivated CD4+ T-cells and pulsed or unpulsed MSCs.

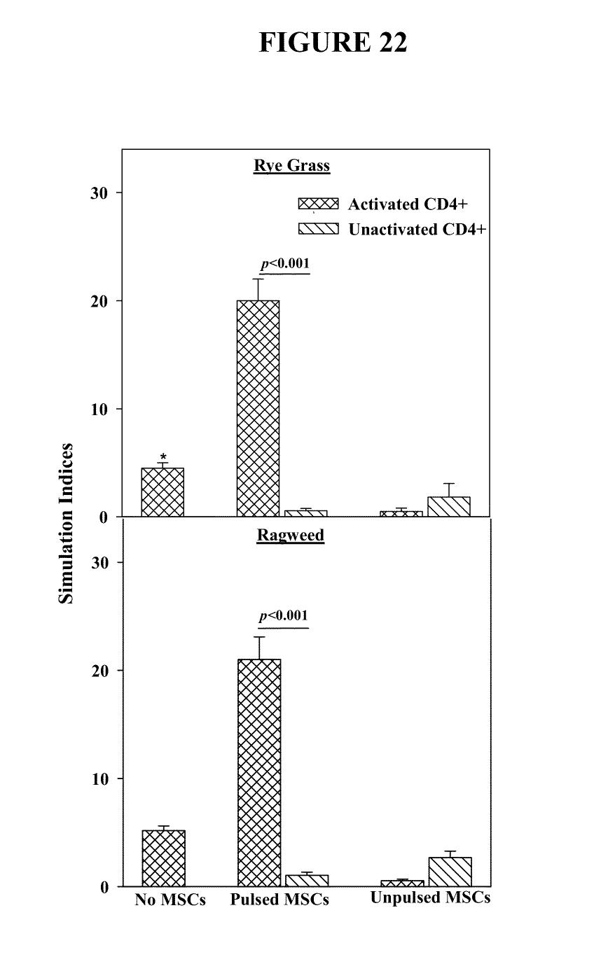

Pre-activated CD4+ T-cells added to antigen-pulsed MSCs resulted in significant (p<0.01) proliferation as compared to unprimed CD4+ T-cells and pulsed MSCs (FIGS. 22A and 22B, middle group). Interestingly, when unpulsed MSCs were added to activated CD4+ T-cells, the proliferation was significantly decreased (p<0.05) as compared to unpulsed MSCs/unactivated CD4+ T-cells alone (FIGS. 22A and 22B, right group). This latter observation indicated that the MSCs acted as immune suppressor cells to the activated CD4+ T-cells, consistent with the immune suppressive functions of MSCs within an inflammatory milieu⁴². The suppressive effect of MSCs on activated CD4+ T-cells was specific because similar suppression was not observed for unactivated CD4+ T-cell. Also, pulsed MSCs failed to induce the proliferation of unactivated CD4+ T-cells. Together, the studies with activated T-cells and pulsed MSCs, when combined with the other controls, supported an APC function of MSCs.

MSCs induced the proliferation of PBMCs from AR subjects in the presence of both ragweed and rye grass (FIGS. 19 and 20). In the case of rye grass and MSCs, the response was synergistic (FIG. 19) whereas similar responses to ragweed and MSCs were additive (FIG. 20). The observations in FIG. 22 indicated that the proliferative effects of ragweed-challenged PBMCs in the presence of MSCs were caused by the APC function of MSCs. In summary, the results indicated APC function of antigen-pulsed MSCs and antigen-activated CD4+ T-cells. The results also showed immune suppressive role of unpulsed MSCs when placed in a condition that recapitulated an inflammatory milieu such as preactivated CD4+ T-cells.

### Increased Cytokine Production, and Expressions of MHC-II and CD86

An APC function of MSCs is expected to cause increases in the production of inflammatory cytokines and enhanced expression of MHC-II and the activation marker, CD86. To determine if similar changes occurred in the APC assays, we address this with three AR donors with ragweed allergy in APC cultures. We analyzed the media after 48 h with assays containing antigen pulsed or unpulsed MSCs and activated CD4+ T-cells. Baseline cytokine production was assessed in cultures with PBMCs alone.

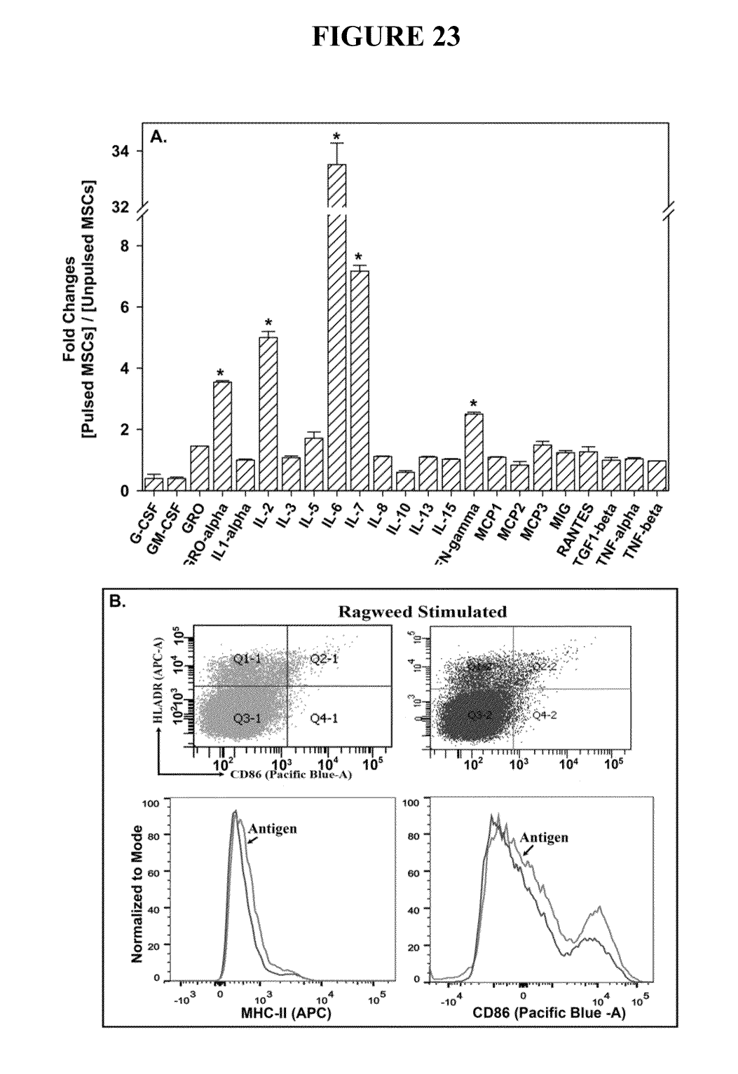

The media were analyzed with cytokine protein arrays in duplicate. The baseline cytokine production was subtracted from the test samples and the resulting densities were normalized to the internal controls. The results were then presented as fold changes of pulsed MSCs/unpulsed MSCs. The results indicated >1.5 fold increases for all cytokines. Particularly, there were significant increases in IL-2, IL-6 and IL-7. IFNγ was increased slightly above the 1.5 fold level (FIG. 23A).

Flow cytometry studied the expressions of MHC-II and CD86 in the antigen-pulsed and unpulsed MSCs from the APC cultures in three independent experiments. We gated the CD 105+ cells (MSCs) if they were negative for CD3 and CD25 and then analyzed this subset for MHC-II and CD86. Both MHC-II and CD86 were increased in the antigen-pulsed MSCs as compared to unpulsed MSCs (FIG. 23B). The shift shown in the histogram was repeated in the two other experiments. The top panels show representative scatter plots, which were represented by overlays of pulsed and unpulsed MSCs in the lower panels. In summary, there were increases in the proinflammatory cytokines, including the chemokine, GRO-1α and, enhanced expressions of MHC-II and the co-stimulatory CD86 on the antigen-pulsed MSCs.

### Blunted APC Function in CIITA Knockdown MSCs

MSCs can express low level of MHC-II and exert APC functions^{22,29,30}. Since CIITA is a master regulator of MHC-II transcription⁴³, we investigated the specificity of APC functions by repeating the APC studies with PBMCs from AR subjects, except with MSCs, knockdown for CIITA. Optimization studies identified clone FI355592 as the most efficient shRNA construct to knockdown CIITA (FIG. 24A, arrow). CIITA knockdown MSCs caused significant (p<0.01) reduction in the proliferation CD4+ T-cells within the APC cultures as compared to untransfected MSCs and vector-transfectants (FIG. 24B). These results, combined with our demonstration that CIITA knockdown decreased HLA-DR expression (FIG. 24A), confirmed the essential role of MSC APC functionality in the observed effects on T-cell proliferation in the context of AR.

### Discussion

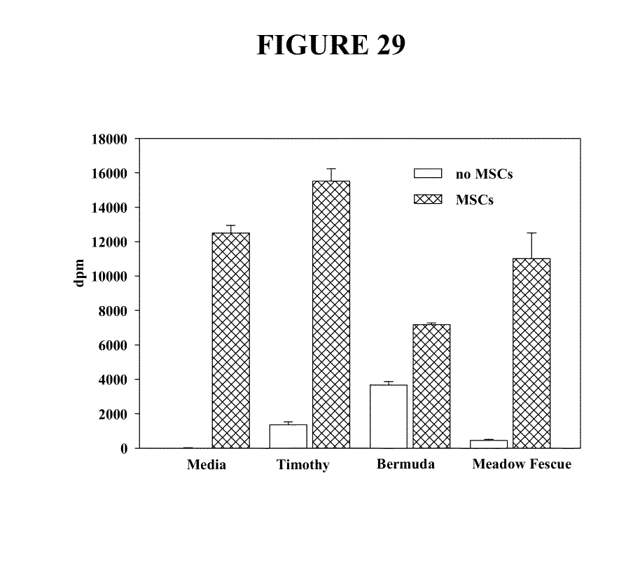

We previously reported that MSCs suppressed the proliferation of PBMCs from patients with allergic asthma and also blunted the maturation of dendritic cells²⁰. We also showed evidence of immune tolerance with repeated exposure of MSCs and allergen to the lymphocytes from subjects with allergic asthma²⁰. Thus, we began this study by assuming that MSCs will suppress the antigen-challenged lymphocytes from AR subjects, without asthma. Our assumption was based on MSCs functioning as immune suppressor cells in an inflammatory milieu as noted for allergic asthma^{20.22}. However, we observed the opposite effects with enhanced proliferation of T-cells when MSCs were added to PBMCs from AR patients (FIGS. 19 and 20). FIGS. 19 and 20 showed the response to ragweed and rye grass; similar effects have been found for other allergens: Timothy, Bermuda and Meadow Fescue (FIG. 29). Interestingly, the PBMCs alone did not show significant differences when challenged with different concentrations of the antigens (FIGS. 27 and 28). However, dose-response effects were noted when the antigens were added together with MSCs (FIGS. 27 and 28). This further confirmed that the increase in proliferation was due to the APC function of MSCs rather than direct interaction with the PBMCs.

In contrast to the studies with AR alone, we tested four subjects with AR and asthma and found that MSCs acted as immune suppressor (FIG. 21). The observations noted for AR with asthma was consistent with the immune suppressor function of MSCs in inflammatory processes^{20,41}. It is expected that the response by antigen-challenged PBMCs from asthmatic subjects with AR induced or directed MSCs to exhibit immune suppressor behavior. Another factor that may contribute to the immune suppressor activity of MSCs under these circumstances is the development of an exacerbated inflammatory milieu. Without wishing to be bound by theory, the response also might be due to a subject's intrinsic sensitivity to allergen, the duration of allergen exposure, and/or the underlying inflammation.

Rye grass stimulation did not show an additive response when PBMCs from AR subjects were stimulated with allogeneic MSCs and antigen stimulation (FIG. 19). Rye grass stimulation in the presence of MSCs resulted in S.I. of about 35 whereas MSCs alone was about 10 and rye grass alone, about 3. Similar studies with ragweed can argue for additive effect (FIG. 20). However, the studies on the APC functions of MSCs with ragweed supported APC effects (FIGS. 22 and 24).

The enhanced proliferation by MSCs was not expected since allogeneic MSCs can exert veto properties^{22.30}. The increased proliferation of the CD4+ T-cells was caused by the APC function of MSC (FIG. 22). This was supported by increases in cytokines, and the expressions of MHC-II and the co-stimulatory CD86 (FIG. 23). The expression of MHC-II requires the expression of the master regulator of transcription, CIITA⁴³. The relationship between CIITA and MHC-II was also needed for MSCs to be APCs (FIG. 24). Although the levels of IFNγ were increased in the APC assay, the increase was not at the same level as IL-2 (FIG. 23). We believe that this relatively modest increase in IFNγ was sufficient to induce T-cell proliferation and differentiation while facilitating sustained MHC-II for APC function³¹.

MSCs presented ragweed to the lymphocytes from AR subjects. However, unpulsed MSCs were able to suppress the proliferation of pre-activated CD4+ cells. This result contrasted with the APC response when pulsed MSCs were added to preactivated PBMCs (FIG. 22, right vs. middle groups, hatched bars). These observations require an expanded discussion because it underscores the possibility that MSCs might be capable of exerting dual immune function within the same milieu. The reason why MSCs can suppress activated CD4+ T-cells is because it gets an opportunity to exert veto function where the activated T-cells mimic a graft versus host type response²². In contrast, antigen-pulsed MSCs are able to present the antigen to activated T-cells.

Qualitative assessment for the sensitivity of the study subjects to the allergens showed some marked differences (Table 3). However, we cannot make an assessment regarding a correlation between sensitivity to the allergen and the responsiveness of MSCs. Consistently, MSCs could increase the proliferation of CD4+ T-cells, indicating APC function.

In an experimental model of AR, MSCs can suppress the immune response of AR^{17,33}. However, the mechanism by which this occurs is yet to be determined. Our in vitro findings, which studied human PBMCs, indicated that MSCs cannot be licensed by AR to become immunosuppressor cells and, such outcome might be dependent on the type of inflammatory disorder. Due to the small number of sample size, we cannot make an assumption on a link between acute sensitivity to the allergens and response of MSCs.

The exacerbated response by MSCs in antigen-challenged PBMCs was a surprise. The clinical translation of such a finding is very important, and suggested that the subjects must be carefully selected. Our studies suggested that the type of response seen, i.e., clinical benefit or worsening of symptoms, following infusion of MSCs might depend on the timing of antigen exposure, perhaps the patient's in vitro sensitivity to the allergen, and mostly if the patients are asthmatic. Given the clinical and economic consequences of poorly controlled AR, it is critical to understand how MSCs will affect a patient's allergic condition. Further investigation in this subject is warranted to understand the underlying mechanisms, and predict clinical response in individuals receiving stem cell therapy.

MSCs exhibited plasticity with regards to the immune response during pollen provocation. Comparing the findings in this study with the veto property of MSCs²², one asks if the particular response of MSCs depends on the level of activation of PBMCs and, perhaps, the in vitro sensitivity to allergen? The findings in this study underscores that one cannot broadly assumes that MSCs will be immune suppressor for inflammatory conditions.

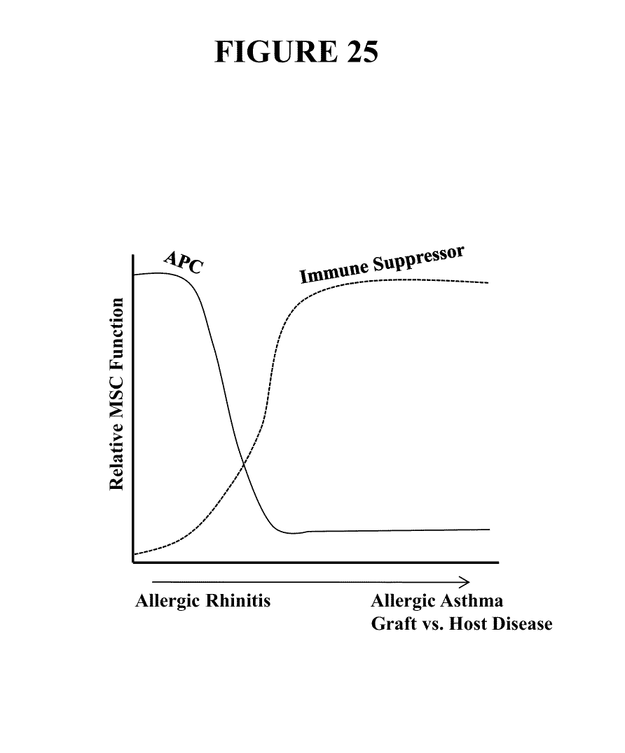

FIG. 25 summarizes the different effects of MSCs in varied inflammatory conditions. The published studies indicate that inflammatory conditions such as allergic asthma and graft versus host disease can be suppressed by MSCs. In contrast, this study shows that similar suppression does not occur for AR. The inflammatory response of allergens to rhinitis and asthma are similar. However, rhinitis could be limited to the lungs whereas asthma might be systemic. Future studies with animal models will be able to address these questions. Also, the degree of inflammation could be important because other studies have shown that if there are inadequate level of cytokines to induce nitric oxide, this could result in inadequate ability of MSCs to be immune suppressor cells⁴⁴.

We expect that CIITA will be within the cytosol of patients with allergic asthma since MSCs suppressed the inflammatory response²⁰.

### Methods

### Reagents

Phosphate Buffered Saline (PBS), pH 7.4 was purchased from Life Technologies (Carlsbad, Calif.), Ficoll Hypaque, DMEM and RPMI 1640 from Sigma (St Louis, Mo.), defined Hyclone fetal calf sera (FCS) from Thermo Fisher Scientific (Waltham, Mass.) and, allergen extracts from Hollister-Stier Laboratories (Spokane, Wash.).

### Study Subjects

Study subjects who met the inclusion criteria and with known AR due to grass or ragweed allergy were included in the study between April and December. The demographics and allergic state of patients are shown in Table 3. The studies used rye grass and ragweed since they are standardized allergens. The Institutional Review Board of University of Medicine and Dentistry of New Jersey, Newark Campus, approved the use of blood from human subjects.

The patient's physician diagnosed AR, based on the following clinical symptoms: nasal congestion, rhinorrhea, sneezing, nasal itching and itchy, watery eyes and skin reactivity to pollen. A positive response on skin prick testing to standardized pollen of grass and ragweed was defined as a wheal response of >3 mm greater as compared to the negative saline control. Subjects were included if skin prick testing was done within one year of enrollment. In addition, patients were enrolled if they were off antihistamine for at least five days. Subjects with significant co-morbid conditions, such as heart disease, atopic dermatitis, immunodeficiency, diabetes, cancer, HIV and pregnancy were excluded. The following exclusion criteria were used: immunotherapy treatment, oral corticosteroids and, immunosuppressants such as methotrexate and azathioprine within 6 months of the date of study. Patients where were using nasal corticosteroid were included in the study. Informed consents were obtained from all study subjects.

Blood samples were drawn at a time when most subjects were asymptomatic or slightly symptomatic and were considered healthy. One subject with asthma was included as a control to evaluate the MSCs, which should exert suppressive effects on the lymphocyte proliferation²⁰. Due to the need to adhere to the approved human subject protocol, e.g., limit on the total amount of blood that was taken for the studies, not all subjects were studied in each assay. The subjects who were used in each assay are stated in the brief description of the figure.

### Isolation of Peripheral Blood Mononuclear Cells (PBMCs)

Approximately 10 mL of blood was obtained from study subjects in heparinized tubes. The PBMCs were immediately isolated by Ficoll-Hypaque gradient separation. The blood was diluted with equal volume of sterile PBS and then added to an equal volume of Histopaque. The buffy coat containing PBMCs was collected and then washed three times in PBS. After the final wash, PBMCs were resuspended at 10⁶/mL RPMI 1640 with 10% FCS.

### Culture of Human MSCs

MSCs were expanded from bone marrow aspirates of healthy volunteers, aged 20-30 years. The use of aspirates was approved by the Institutional Review Board. Each volunteer signed informed consent. The method was previously described²². Briefly, unfractionated aspirates were diluted in DMEM containing 10% fetal calf sera and then placed in vacuum gas plasma-treated plates (BD Falcon; Franklin Lakes, N.J.). The initial plating of whole BM aspirates prevented the loss of endogenous MSCs. After 3 days of incubation, Ficoll Hypaque density gradient was used to separate the mononuclear fraction from the red blood cells and neutrophils. The reason for removing the red blood cells was to avoid toxicity to the adherent MSCs, caused by red cell lysis. The mononuclear cells were replaced in the original culture dishes. At weekly intervals, 50% of the media was replaced with fresh media. The adherent cells were serially passaged and after four cell passages, the adherent cells were symmetric, CD14−, CD29+, CD44+, CD34−, CD45−, CD105+, prolyl-4-hydroxylase-.

The method described above selected a population of pluripotent MSCs that expressed low level of MHC-II. The expanded MSCs formed functional peptidergic and dopaminergic neurons as well as differentiation along adipogenic, osteogenic and chondrogenic lineages^{22,45}. We compared different tissue culture surfaces and obtained high efficiency of pluripotent MSCs with plasma-treated surfaces.

We have determined that the surface and the type of culture dish (flask versus petri dish) are important in the doubling times for the MSCs. Some laboratories used platelet rich plasma to expand MSCs. Since platelets are a rich source of TGFβ1, we did not use this method because this might include a bias into MSCs with immune suppressive functions. Nonetheless, we compared MSCs cultured in petri dishes (Falcon 3003) and corning tissue culture flask with the same media. The MSCs in the petri dishes showed a longer doubling time, but both sources showed similar responses with respect to the functional studies.

### Proliferation Assays

Cell proliferation was based on ³H-TdR incorporation as described²². PBMCs were resuspended at 10⁶/mL in RPMI 1640 containing 10% FCS and then stimulated with allergen. The allergen was selected from the subject's known allergies (Table 3). Pollen (Hollister-Stier) was added at 5 μL/mL using the following: standardized Rye Grass (Lot # E08L1341); Timothy Grass (Lot #F08L1362); Bermuda (Lot #G09L7298); Meadow Fescue (Lot #C08J0534); short Ragweed (Lot #E10F0143). Except for ragweed, the stock concentrations of all allergens were 10,000 BAU/mL. The stock concentration of ragweed was 1:20 weight/volume with an Amba1 content of 191. The optimal concentrations for each allergen were determined in titration assays for each subject in dose-response curves. The concentrations, at peak proliferation, were selected for all studies. The concentrations of all allergens except ragweed, presented as μL/mL, are equivalent to 50 BAU/mL.

PBMCs and MSCs were added at 50:1 ratios in wells of 96-well tissue culture plates. This was achieved by diluting the PBMCs at 10⁶/ml and MSCs, at 2×10⁴/ml. The MSCs were irradiated with 2000 rads or non-irradiated. The radiation was delivered with a cesium source as described²². The cell mixture contained the respective antigen at the concentration, stated above. The following parallel cultures were performed: PBMCs with antigen in the absence of MSCs; PBMCs and MSCs without antigen. After 48 h, each well was pulsed with 1 μCi/mL of ³H-TdR. After 16 h, the cells were harvested on glass fiber filters to study radioactive incorporation in a liquid scintillation counter (Beckman; Fullerton, Calif.). The stimulation indices (S.I.) were calculated as disintegration per min (dpm) of experimental points/dpm of unstimulated PBMCs. We used dpm instead of counts per min (cpm) to account for the efficiency of the scintillation counters used to count the incorporation of ³H-TdR.

### Immunoprecipitation/Western Blot

Immunoprecipitation of Class II, major histocompatibility complex, transactivator (CIITA) was performed with cytoplasmic and nuclear cell extracts using the Protein G-Agarose kit (Roche Applied Bioscience, Indianapolis, Ind.). Briefly, the extracts were incubated with anti-CIITA (Santa Cruz, Santa Cruz, Calif.) at 1/500 final dilution at 4° C. overnight. After this, the reactions were incubated with protein-G agarose (1/50) at 4° C. for 4 h on a rocking platform. The reactions were centrifuged at 4° C., 12000×g for 15 min, and the pellets were washed once with 1×PBS and then resuspended in 1× sample buffer containing 0.5% β-ME. The extracts (20 μg of total protein) were analyzed by western blots.

The samples were electrophoresed on a 12% Mini-PROTEAN Precast Gel (Bio-RAD, Hercules, Calif.) and then transferred to polyvinylidene difluoride membranes (PerkinElmer, Whaltham, Mass.). The membranes were incubated with anti-CIITA at 1/500 dilution, 4° C. overnight; washed; incubated with HRP-conjugated goat anti-mouse IgG (1/1000) for 2 h at 4° C. HRP was developed with chemiluminescence detection reagents (Thermo Scientific). The molecular weight was determined with SeeBlue^{M} plus 2 Pre-stained standards (Life Technology).

Western blot for HLA-DR used rabbit polyclonal anti-HLA-DRa; ribosomal protein used goat polyclonal antibody; Acetyl-Histone H3 used rabbit polyclonal IgG. All antibodies were purchased from (Lake Placid); β-actin used murine monoclonal IgG (Sigma).

### CIITA Knockdown

CIITA shRNA were purchased from Origene (Rockville, Md.). Four different inserts were tested to identify the insert that could efficiently knockdown CIITA. The inserts were ligated in pRFP-C-RS under the control of U6 promoter and were assigned the following by Origene: FI355589, FI355590, FI355591 and FI355592. The plasmid and vector without insert were transfected in MSCs as described using Effectene Transfection Reagent Kit (Qiagen)³⁰. At 72 h and one week after transfection, western blots for CIITA indicated efficient knockdown by the FI355592 plasmid. All assays were done with MSCs transfected with FI355592.

Antigen Presenting Cell (APC) assay

Day 1, Cell Activation:

PBMCs (5×10⁶/ml) were incubated with optimum dose of ragweed or rye grass (5 μL/ml). Unactivated cells contained only media. Day 4, Pulsing: MSCs (2×10⁴/ml) were incubated for 24 h with the same amount of the allergens. Unpulsed MSCs omitted antigen. Day 5, Enrichment of CD4⁺ T-cells: CD4+ cells were enriched by negative selection of other immune cell subsets. PBMCs (10⁶/ml) were incubated with a cocktail of antibodies: CD3, CD8, CD11, CD56, CD20, each at 1/200 final dilution. After one h of incubation on ice, cells were washed with PBS and resuspended in 0.5 mL of PBS and 100 μL of Dynabead goat anti-mouse IgG (Invitrogen). The Dynabead-coupled cells were removed with a magnetic separator. The negative population was analyzed for CD4 by flow cytometry and the result indicated >90% labeling with anti-CD4. Day 5, Assay: Pulsed MSCs were resuspended in DMEM with 10% FCS at 10⁶/ml and then subjected to 2000 rads of γ-irradiation. Irradiation rendered the cells in cycling quiescence, but metabolically active. CD4⁺ enriched cells (4×10⁴/ml) were added to 50, 10², 10³ or 10⁴/ml to the γ-irradiated MSCs. After 24 h, cells were pulsed with 1 μCi of [methyl-³H]-TdR/well. After 16 h, cells were harvested, analyzed for radioactive incorporation and the simulation indices were calculated by dividing the dpm of experimental points by dpm of unactivated CD4⁺ T-cells.

### Flow Cytometry

Flow cytometry MHC-II and CD86 was performed with multi-color flow cytometry and the following antibodies: V450-CD86 and CD105 (Becton Dickinson, Franklin Lakes, N.J.), phycoerythrin-CD14 (Becton Dickinson), allophycocyanin-HLA-DR (Caltag Laboratories, Burlingame, Calif.), CD3 and CD25 from a Human Regulatory T Cell Staining Kit from e-Bioscience (San Diego, Calif.). Non-specific labelings used fluorochrome conjugated isotype from Becton Dickinson. Positive control BD Compbeads (BD Bioscience) were labeled with the all fluorochrome-tagged antibodies. After labeling, cells were analyzed with the LSRII system (BD Biosciences, San Jose, Calif.). The cells were gated on those positive for CD105 and negative for CD3 and CD25. The data were analyzed with the FACSCalibur system (BD Biosciences).

### Cytokine Array

Cytokine production by PBMCs, with pulsed and unpulsed MSCs was assessed using the Human Cytokine Antibody Array 1 (RayBiotech; Norcross, Ga.), as per manufacturer's instruction, also previously described²². Briefly, the media were collected after 48 h for cytokine determination. The media from cultures containing PBMCs alone were assessed for background cytokine. The values were subtracted from the experimental. The densities of spots were quantitated with UN-SCAN-IT densitometry software (Silk Scientific; Orem, Utah). Cytokines were normalized to internal positive controls and presented as fold change relative to an internal control, arbitrarily assigned a value of 1. After this, the data were presented as fold changes of cultures with pulsed MSCs/unpulsed MSCs.

### Statistical Analysis

Data were analyzed using analysis of variance and Tukey-Kramer multiple comparisons test. A P value of <0.05 was considered significant.

TABLE 3
Demographics of enrolled subjects

| Subject | Age | Ethnicity | Skin Prick Test |
|---|---|---|---|
| S1-S4 | 30-35 | Hispanic | Rye grass (2+); Positive grasses, |
| | | | trees, mold |
| S5-S8 | 37-40 | Asian | Rye grass (2+); Positive to trees, |
| | | | dust mite |
| S9-S12 | 20-25 | Hispanic | Ragweed (4+); Positive to trees, |
| | | | cockroach, cat pelt |
| S13-S16 | 23-30 | African | Ragweed (4+) |
| | | American | |
| S17 | 27 | Asian | Ragweed (4+), Bermuda grass (+) |
| S18 | 30 | Hispanic | Asthma, Rye grass (4+); Ragweed |
| | | | (4+); Positive to grasses and |
| | | | other weeds, dust mite, and cat |
| S19-21 | 27-30 | African | Asthma, Rye grass (2+); Ragweed |
| | | American | (4+); Dustmite (−) |
| S22 | 30 | Hispanic | Ragweed 3+; Positive to trees, |
| | | | grass (−) |

### References for Example 3

1. Giordano A, Galderisi U, Marino I R. From the laboratory bench to the patient's bedside: An update on clinical trials with mesenchymal stem cells. J Cell Physiol 2007; 211: 27-35.
2. Helmy K Y, Patel S A, Silverio K, Pliner L, Rameshwar P. Stem cells and regenerative medicine: accomplishments to date and future promise. Ther Deliv 2010; 1: 693-705.
3. Greco S J, Rameshwar P. Mesenchymal stem cells in drug/gene delivery: implications for cell therapy. Ther Deliv 2012; 3: 997-1004.
4. Caplan A I, All MSCs are pericytes? Cell Stem Cell 2008; 3: 229-230.
5. Boregowda S V, Phinney D G. Therapeutic applications of mesenchymal stem cells: current outlook. BioDrugs 2012; 26: 201-208.
6. Patel S A, Meyer J R, Greco S J, Corcoran K E, Bryan M, Rameshwar P. Mesenchymal Stem Cells Protect Breast Cancer Cells through Regulatory T Cells: Role of Mesenchymal Stem Cell-Derived TGF-{beta}. J Immunol 2010; 184: 5885-5894.
7. Rameshwar P: Breast cancer cell dormancy in bone marrow: potential therapeutic targets within the marrow microenvironment. Expert Rev Anticancer Ther 2010; 10:129-132.
8. Comsa S, Ciuculescu F, Raica M. Mesenchymal stem cell-tumor cell cooperation in breast cancer vasculogenesis. Mol Med Report 2012; 5: 1175-1180.
9. Momin E N, Vela G, Zaidi H A, Quinones-Hinojosa A. The Oncogenic Potential of Mesenchymal Stem Cells in the Treatment of Cancer: Directions for Future Research. Curr Immunol Rev 2010; 6: 137-148.
10. Feng B, Chen L: Review of mesenchymal stem cells and tumors: executioner or coconspirator? Cancer Biother Radiopharm 2009; 2 4: 717-721.
11. Greco S J, Rameshwar P. Microenvironmental considerations in the application of human mesenchymal stem cells in regenerative therapies. Biologics 2008; 2: 699-705.
12. Riggi N, Suva M L, De V C, Provero P, Stehle J C, Baumer K et al. EWS-FLI-1 modulates miRNA145 and SOX2 expression to initiate mesenchymal stem cell reprogramming toward Ewing sarcoma cancer stem cells. Genes Dev 2010; 24: 916-932.
13. De B A, Narine K, De N R, Marcel M, Bracke M, De W O. Resident and bone marrow-derived mesenchymal stem cells in head and neck squamous cell carcinoma. Oral Oncol 2010; 46: 336-342.
14. Corcoran K E, Fernandes H, Bryan M, Taborga M, Srinivas V, Packman K, Rameshwar P. Mesenchymal stem cells in early entry of breast cancer into bone marrow. PLoS One 2008; 3: e2563, doi: 10.1371/journal.pone.0002563.
15. Mishra P J, Mishra P J, Humeniuk R, Medina D J, Alexe G, Mesirov J P, Ganesan S, Glod J W, Banerjee D. Carcinoma-Associated Fibroblast-Like Differentiation of Human Mesenchymal Stem Cells. Cancer Res 2008; 68: 4331-4339.
16. Patel S A, Ramkissoon S H, Bryan M, Pliner L F, Dontu G, Patel P S et al. Delineation of breast cancer cell hierarchy identifies the subset responsible for dormancy. Sci Rep 2012; 2. doi:10.1038/srep00906
17. Cho K S, Park H K, Park H Y, Jung J S, Jeon S G, Kim Y K et al. IFATS Collection: Immunomodulatory Effects of Adipose Tissue-Derived Stem Cells in an Allergic Rhinitis Mouse Model. Stem Cells 2009; 27: 259-265.
18. Vaes B, Van't H of W, Deans R, Pinxteren J. Application of MultiStem® Allogeneic Cells for Immunomodulatory Therapy: Clinical Progress and Pre-Clinical Challenges in Prophylaxis for Graft Versus Host Disease. Front Immunol 2012; 3: 345.
19. Iyer S S, Co C, Rojas M. Mesenchymal stem cells and inflammatory lung diseases. Panminerva Med 2009; 51: 5-16.
20. Kapoor S, Patel S A, Kartan S, Axelrod D, Capitle E, Rameshwar P. Tolerance-like mediated suppression by mesenchymal stem cells in patients with dust mite allergyΓ [symbol] ôinduced asthma. J Allergy Clin Immunol 2012; 129: 1094-1101.
21. Ryan J M, Barry F, Murphy J M, Mahon B P. Interferon-gamma does not break, but promotes the immunosuppressive capacity of adult human mesenchymal stem cells. Clin Exp Immunol 2007; 149: 353-363.
22. Potian J A, Aviv H, Ponzio N M, Harrison J S, Rameshwar P. Veto-Like Activity of Mesenchymal Stem Cells: Functional Discrimination Between Cellular Responses to Alloantigens and Recall Antigens. J Immunol 2003; 171: 3426-3434.
23. Patel S A, Sherman L, Munoz J, Rameshwar P. Immunological properties of mesenchymal stem cells and clinical implications. Arch Immunol Ther Exp 2008; 56: 1-8.
24. Nauta A J, Fibbe W E. Immunomodulatory properties of mesenchymal stromal cells. Blood 2007; 110: 3499-3506.
25. Marigo I, Dazzi F. The immunomodulatory properties of mesenchymal stem cells. Sem Immunopathol 2011; 33: 593-602.
26. Ren G, Zhang L, Zhao X, Xu G, Zhang Y, Roberts A I et al. Mesenchymal Stem Cell-Mediated Immunosuppression Occurs via Concerted Action of Chemokines and Nitric Oxide. Cell Stem Cell 2008; 2: 141-150.
27. Salem H K, Thiemermann C. Mesenchymal Stromal Cells: Current Understanding and Clinical Status. Stem Cells 2010; 28: 585-596.
28. Stagg J, Galipeau J. Immune plasticity of bone marrow-derived mesenchymal stromal cells. Handb Exp Pharmacol 2007; 180: 45-66.
29. Stagg J, Pommey S, Eliopoulos N, Galipeau J. Interferon-gamma-stimulated marrow stromal cells: a new type of nonhematopoietic antigen-presenting cell. Blood 2006; 107: 2570-2577.
30. Chan J L, Tang K C, Patel A P, Bonilla L M, Pierobon N, Ponzio N M et al. Antigen-presenting property of mesenchymal stem cells occurs during a narrow window at low levels of interferon-gamma. Blood 2006; 107: 4817-4824.
31. Tang K C, Trzaska K A, Smirnov S V, Kotenko S V, Schwander S K, Ellner J J et al. Down-Regulation of MHC II in Mesenchymal Stem Cells at High IFN-gamma Can Be Partly Explained by Cytoplasmic Retention of CIITA. J Immunol 2008; 180: 1826-1833.
32. Francois M, Romieu-Mourez R, Stock-Martineau S, Boivin M N, Bramson J L, Galipeau J. Mesenchymal stromal cells cross-present soluble exogenous antigens as part of their antigen-presenting cell properties. Blood 2009; 114: 2632-2638.
33. Goodwin M, Sueblinvong V, Eisenhauer P, Ziats N P, LeClair L, Poynter M E et al. Bone Marrow-Derived Mesenchymal Stromal Cells Inhibit Th2-Mediated Allergic Airways Inflammation in Mice. Stem Cells 2011; 29: 1137-1148.
34. Nemeth K, Keane-Myers A, Brown J M, Metcalfe D D, Gorham J D, Bundoc V G et al. Bone marrow stromal cells use TGF-beta to suppress allergic responses in a mouse model of ragweed-induced asthma. Proc Natl Acad Sci 2010; 107: 5652-5657.
35. Settipane R A. Demographics and epidemiology of allergic and nonallergic rhinitis. Allergy Asthma Proc 2001; 22: 185-189.
36. Commins S P, Borish L, Steinke J W. Immunologic messenger molecules: Cytokines, interferons, and chemokines. J Allergy Clin Immunol 2010; 125: S53-S72.
37. Meltzer E O, Bukstein D A: The economic impact of allergic rhinitis and current guidelines for treatment. Ann Allergy, Asthma Immunol 2011; 106: S12-S16.
38. Dazzi F, Lopes L, Weng L. Mesenchymal stromal cells: a key player in ‘innate tolerance’? Immunology 2012; 137: 206-213.
39. Crop M J, Baan C C, Korevaar S S, Ijzermans J N M, Weimar W, Hoogduijn M J. Human Adipose Tissue-Derived Mesenchymal Stem Cells Induce Explosive T-Cell Proliferation. Stem Cells Dev 2010; 19: 1843-1853.
40. Newman R E, Yoo D, LeRoux M A, Danilkovitch-Miagkova A. Treatment of inflammatory diseases with mesenchymal stem cells. Inflamm Allergy Drug Targets 2009; 8: 110-123.
41. Uccelli A, Pistoia V, Moretta L. Mesenchymal stem cells: a new strategy for immunosuppression? Trends Immunol 2007; 28: 219-226.
42. Siegel G, Schafer R, Dazzi F. The immunosuppressive properties of mesenchymal stem cells. Transplantation 2009; 87: S45-S49.
43. Wright K L, Ting J P Y. Epigenetic regulation of MHC-II and CIITA genes. Trends Immunol 2006; 27: 405-412.
44. Li W, Ren G, Huang Y, Su J, Han Y, Li J et al. Mesenchymal stem cells: a double-edged sword in regulating immune responses. Cell Death Differ 2012; 19: 1505-1513.
45. Trzaska K, Rameshwar P. Dopaminergic Neuronal Differentiation Protocol for Human Mesenchymal Stem Cells. In: Vemuri M, Chase L G, Rao M S (eds) Mesenchymal Stem Cell Assays and Applications. Methods in Molecular Biology. Humana Press, Totowa, N.J. 2011, pp 295-303.

Each of the references cited herein is incorporated by reference in its entirety or as its relevance would be apparent from the context of its usage.

From the disclosure herein it will be appreciated that, although specific embodiments of the disclosure have been described herein for purposes of illustration, various modifications may be made without deviating from the spirit and scope of the disclosure.

What is claimed is:

1. A method of treating an inflammatory condition in an asthmatic subject comprising administering an effective dose of a cell exhibiting the phenotype of a mesenchymal stem cell.

2. The method according to claim 1 wherein the inflammatory condition is systemic lupus erythematosis, rheumatoid arthritis, inflammatory bowel disease, Crohn's disease, autoimmune enteropathy, colitis, neonatal colitis, neonatal necrotizing enterocolitis, pouchitis, or an allergic reaction.

3. The method according to claim 2 wherein the inflammatory condition is an allergic reaction in the subject.

4. The method according to claim 1 wherein the cell is a mesenchymal stem cell.

5. The method according to claim 4 wherein the mesenchymal stem cell is an autologous cell.

6. The method according to claim 4 wherein the mesenchymal stem cell is obtained from bone marrow, adipose tissue, placenta, fetal organ, or amniotic fluid.

7. The method according to claim 3 wherein the allergic reaction is a reaction to dust mite.

8. The method according to claim 7 wherein the dust mite is Dermatophagoides farinae or Dermatophagoides pteronyssinus.

9. The method according to claim 3 wherein the allergic reaction is a reaction to a food allergen, a drug allergen, or an environmental allergen.

10. The method according to claim 9 wherein the food allergen is fruit, garlic, oats, meat, a milk product, peanut, fish, shellfish, soy, a tree nut, wheat, gluten, egg, a sulfite, carbohydrate, or chocolate, or the drug allergen is tetracycline, dilantin, tegretol, penicillin, a cephalosporin, a sulfonamide, a non-steroidal anti-inflammatory drug (NSAID), an intravenous contrast dye, or a local anesthetic, or the environmental allergen is pollen, pet dander, an insect bite, an insect sting, mold, perfume, a cosmetic, semen, latex, metal, formaldehyde, tobacco, or a photographic developer.

11. A method of inducing tolerance to an allergen in an asthmatic subject comprising:
(a) administering an effective dose of mesenchymal stem cells; and
(b) delivering at least one sub-stimulatory dose of an allergen to the subject, thereby inducing tolerance to the allergen in the subject.

12. The method according to claim 11 wherein the sub-acute dose of an allergen produces 0.125-5.0 μg/ml trough concentration in serum.

13. The method according to claim 11 wherein the mesenchymal stem cell is an autologous cell.

14. The method according to claim 11 wherein the mesenchymal stem cell is obtained from bone marrow, adipose tissue, placenta, fetal organ, or amniotic fluid.

15. The method according to claim 11 wherein the allergic reaction is a reaction to dust mite.

16. The method according to claim 15 wherein the dust mite is Dermatophagoides farinae or Dermatophagoides pteronyssinus.

17. A method of assessing a subject for amenability to allergy treatment with a cell exhibiting the phenotype of a mesenchymal stem cell comprising:
(a) contacting a non-asthmatic subject with at least one dose of an allergen; and
(b) determining the response of the subject to the allergen, wherein an acute allergic reaction is indicative of an individual amenable to allergy treatment with the cell.


SEQUENCE LISTING (4 sequences)

SEQ ID NO: 1 - DNA, 29 bp, Artificial Sequence
  Synthetic Primer
tcaggcagcagaggagaagttcaccatcg

SEQ ID NO: 2 - DNA, 29 bp, Artificial Sequence
  Synthetic Primer
actgcgaccagttcagcaggctgttgtgt

SEQ ID NO: 3 - DNA, 29 bp, Artificial Sequence
  Synthetic Primer
ccaggcatacgtgatgcgctactttgaga

SEQ ID NO: 4 - DNA, 29 bp, Artificial Sequence
  Synthetic Primer
acctgaccgcgttctgctcatcctagacg